(19) United States
(12) Patent Application Publication
(10) Pub. No.: US 2019/0070280 A1
(43) Pub. Date: Mar. 7, 2019

(54) ONCOLYTIC VIRUS AND CHECKPOINT INHIBITOR COMBINATION THERAPY

(71) Applicant: TURNSTONE LIMITED PARTNERSHIP, TORONTO (CA)

(72) Inventors: BRIAN LICHTY, BRANTFORD (CA); JOHN BELL, OTTAWA (CA)

(21) Appl. No.: 16/059,914

(22) Filed: Aug. 9, 2018

Related U.S. Application Data
(63) Continuation of application No. 16/069,136, filed on Jul. 10, 2018.
(60) Provisional application No. 62/277,352, filed on Jan. 11, 2016.

(51) Int. Cl. A61K 39/00; A61K 39/395; A61P 35/00

(52) U.S. Cl. CPC A61K 39/00119; A61K 39/3955; A61K 2039/5256; A61K 2039/86; A61P 35/00

(57) ABSTRACT

The present invention pertains to a combination for simultaneous, separate or sequential use which comprises (a) an oncolytic virus and (b) a checkpoint inhibitor and to its use for the treatment of cancer.

### BACKGROUND OF THE INVENTION

### Field of the Invention

This invention relates generally to virology and medicine. In certain aspects the invention relates to combination therapy with oncolytic viruses, particularly oncolytic rhabdoviruses and checkpoint inhibitors for the treatment of cancer.

### Background

Oncolytic viruses specifically infect, replicate in, and kill malignant cells leaving normal tissues unaffected. Several oncolytic viruses have reached advanced stages of clinical evaluation for the treatment of a variety of neoplasms.

Rhabdoviruses displaying oncolytic activity have been described, including vesicular stomatitis virus (VSV) and Maraba virus. The inherent oncotropism of these viruses can be further enhanced by mutations which increase the sensitivity of the virus to host immune responses.

The efficacy of oncolytic viruses depends not only on their cytolytic activity but also on their ability to stimulate antitumoral immunity. One approach to enhancing the clinical effectiveness of oncolytic viruses is to express a tumor antigen from the virus. Thus, it has been demonstrated that VSV engineered to express a tumor antigen can be used as an oncolytic viral immunotherapy. The antitumoral efficacy of VSV expressing a tumor antigen has been shown to be enhanced by first administering the tumor antigen prior to the engineered VSV to prime antitumoral immunity and subsequently administering the oncolytic virus expressing the same tumor antigen to boost the existing antitumoral immunity (Bridle et al., Mol. Ther., 18(8):1430-1439 (2010)).

Further approaches to enhance the efficacy of oncolytic viruses are needed.

### SUMMARY OF THE INVENTION

The present inventors have discovered that co-administration of an oncolytic virus and an immune checkpoint inhibitor to clinically relevant cancer models results in a surprising increase in the stimulation of antigen-specific T lymphocytes concomitant with a significant survival benefit relative to administration of either agent alone. Accordingly, in several embodiments, the present application provides a combination therapy for use in the treatment and/or prevention of cancer and/or the establishment of metastases in a mammal and/or for use in initiating, enhancing or prolonging an anti-tumor response in a mammal comprising co-administering to the mammal (i) an oncolytic virus in combination with (ii) one or more immune checkpoint inhibitors. In certain aspects, co-administration of an oncolytic virus and immune checkpoint inhibitor to a subject with cancer provides an enhanced and even synergistic anti-tumor immunity compared to either treatment alone. In related aspects, the anti-tumor effects of the combination therapy persist even after clearance of the virus and may extend to one or more non-infected tumors. In other related aspects, a method for enhancing, potentiating or prolonging the effects of a checkpoint inhibitor or enabling the toxicity or dose or number of treatments of a checkpoint inhibitor to be reduced comprising administering to a mammal in need thereof (i) an oncolytic virus in combination with (ii) one or more immune checkpoint inhibitors.

In some embodiments, the oncolytic virus according to the combination therapy is a replication competent oncolytic rhabdovirus. Such oncolytic rhabdovirusus include, without limitation, wild type or genetically modified Arajas virus, Chandipura virus, Cocal virus, Isfahan virus, Maraba virus, Piry virus, Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bimbo virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Manitoba virus, Marco virus, Nasoule virus, Navarro virus, Ngaingan virus, Oak-Vale virus, Obodhiang virus, Oita virus, Ouango virus, Parry Creek virus, Rio Grande cichlid virus, Sandjimba virus, Sigma virus, Sripur virus, Sweetwater Branch virus, Tibrogargan virus, Xiburema virus, Yata virus, Rhode Island, Adelaide River virus, Berrimah virus, Kimberley virus, or Bovine ephemeral fever virus. In some preferred embodiments, the oncolytic rhabdovirus is a wild type or recombinant vesiculovirus. In other preferred embodiments, the oncolytic rhabdovirus is a wild type or recombinant VSV, Farmington, Maraba, Carajas, Muir Springs or Bahia grande virus, including variants thereof. In particularly preferred embodiments, the oncolytic rhabdovirus is a VSV or Maraba rhabdovirus. In other particularly preferred embodiments, the oncolytic rhabdovirus is a VSV or Maraba rhabdovirus comprising one or more genetic modifications that increase tumor selectivity and/or oncolytic effect of the virus.

In related embodiments, the oncolytic virus according to the combination therapy is engineered to express one or more tumor antigens, such as those mentioned in paragraphs [0071]-[0082] of WIPO publication no. WO 2014/127478 and paragraph [0042] of U.S. Patent Application Publication No. 2012/0014990, the contents of both of which are incorporated herein by reference. In preferred embodiments, the oncolytic virus is an oncolytic rhabdovirus (e.g. VSV or Maraba strain) that expresses MAGEA3, Human Papilloma Virus E6/E7 fusion protein, human Six-Transmembrane Epithelial Antigen of the Prostate protein, or Cancer Testis Antigen 1, or a variant thereof. In particularly preferred embodiments, the oncolytic virus is an oncolytic rhadovirus selected from Maraba MG1 and VSVdelta51 that expresses MAGEA3, Human Papilloma Virus E6/E7 fusion protein, human Six-Transmembrane Epithelial Antigen of the Prostate protein, or Cancer Testis Antigen 1, or a variant thereof.

In some aspects, a combination therapy for treating and/or preventing cancer in a mammal is provided comprising co-administering to the mammal (i) an oncolytic rhabdovirus (e.g. VSVdelta51 or Maraba MG1) expressing a tumor antigen to which the mammal has a pre-existing immunity selected from MAGEA3, Human Papilloma Virus E6/E7 fusion protein, human Six-Transmembrane Epithelial Antigen of the Prostate protein, or Cancer Testis Antigen 1, or a variant thereof and (ii) a checkpoint inhibitor (e.g. a monoclonal antibody against CTLA4 or PD-1/PD-L1). In preferred embodiments, the pre-existing immunity in the mammal is established by vaccinating the mammal with the tumor antigen prior to administration of the oncolytic virus. In related embodiments, a first dose of checkpoint inhibitor is administered prior to a first dose of oncolytic rhabdovirus expressing the tumor antigen and subsequent doses of checkpoint inhibitor may be administered after a first (or second, third and so on) of oncolytic rhabdovirus expressing the tumor antigen.

In another aspect of the combination described herein, the oncolytic rhabdovirus expresses the checkpoint inhibitor (e.g. the oncolytic rhabodvirus expresses a single chain antibody against a checkpoint inhibitor protein) and optionally also expresses a tumor-associated antigen as herein described.

The oncolytic virus of the combination may be administered as one or more doses of 10, 100, 10³, 10⁴, 10⁵, 10⁶, 10⁷, 10⁸, 10⁹, 10¹⁰, 10¹¹, 10¹², 10¹³, 10¹⁴, or more viral particles (vp) or plaque forming units (pfu). In preferred embodiments, the oncolytic virus is an oncolytic rhabdovirus (e.g. wild type or genetically modified VSV or Maraba optionally expressing one or more tumor antigens) and is administered to a human with cancer as one or more dosages of 10⁶-10¹⁴ pfu, 10⁶-10¹² pfu, 10⁸-10¹⁴ pfu, 10⁸-10¹² or 10¹⁰-10¹² pfu or any range therebetween. Administration can be by intraperitoneal, intravenous, intra-arterial, intramuscular, intradermal, subcutaneous, or intranasal administration. In preferred embodiments, the oncolytic virus is administered systemically, particularly by intravascular administration, which includes injection, perfusion and the like.

In some aspects, a checkpoint inhibitor of the combination is a biologic therapeutic or small molecule. In another aspect, the checkpoint inhibitor is a monoclonal antibody, a humanized antibody, a human antibody, a fusion protein or a combination thereof. In a further aspect, the checkpoint inhibitor inhibits a checkpoint protein including without limitation cytotoxic T-lymphocyte antigen-4 (CTLA4), programmed cell death protein 1 (PD-1) and its ligands PD-L1 and PD-L2, B7-H3, B7-H4, herpesvirus entry mediator (HVEM), T cell membrane protein 3 (TIM3), galectin 9 (GALS), lymphocyte activation gene 3 (LAG3), V-domain immunoglobulin (Ig)-containing suppressor of T-cell activation (VISTA), Killer-Cell Immunoglobulin-Like Receptor (KIR), B and T lymphocyte attenuator (BTLA), T cell immunoreceptor with Ig and ITIM domains (TIGIT) or a combination thereof. In an additional aspect, the checkpoint inhibitor interacts with a ligand of a checkpoint protein including without limitation CTLA4, PD-1, B7-H3, B7-H4, HVEM, TIM3, GALS, LAG3, VISTA, KIR, BTLA, TIGIT or a combination thereof.

In some preferred embodiments, the oncolytic virus (e.g. oncolytic rhabdovirus) is co-administered with a CTLA4 checkpoint inhibitor. CTLA4 checkpoint inhibitors include, without limitation, monoclonal antibodies such as Ipilimumab (Yervoy®; BMS) and Tremelimumab (AstraZeneca/MedImmune).

In other preferred embodiments, the oncolytic virus (e.g. oncolytic rhabdovirus) is co-administered with an inhibitor of PD-1 or its ligand (PD-L1). PD-1/PD-L1 checkpoint inhibitors include, without limitation, monoclonal antibodies against PD-1 such as Nivolumab (Opdivo®; Bristol-Myers Squibb; code name BMS-936558), Pembrolizumab (Keytruda®) and Pidilizumab, anti-PD-1 fusion proteins such as AMP-224 (composed of the extracellular domain of PD-L2 and the Fc region of human IgG1), and monoclonal antibodies against PD-L1 such as BMS-936559 (MDX-1105), Atezolizumab (Genentech/Roche; MPDL3280A), Durvalumab (AstraZeneca/MedImmune; MEDI4736) and Avelumab (Merck KGaA).

The oncolytic virus (e.g. oncolytic rhabdovirus) and immune checkpoint inhibitor are administered simultaneously or sequentially to the mammal in need thereof and may be administered as part of the same formulation or in different formulations. In preferred embodiments, treatment with the oncolytic virus is initiated prior to initiating treatment with the checkpoint inhibitor.

Cancers to be treated according to the combination described herein include, without limitation, leukemia, acute lymphocytic leukemia, acute myelocytic leukemia, myeloblasts promyelocyte, myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung (thoracic) cancer (including small cell lung cancer, squamous non-small cell lung cancer and non-squamous non-small cell lung cancer)), melanoma (including metastatic melanoma), neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system. In some preferred embodiments, the cancer to be treated is selected from squamous or non-squamous non-small cell lung cancer (NSCLC), breast cancer (e.g. hormone refractory metastatic breast cancer), head and neck cancer (e.g. head and neck squamous cell cancer), metastatic colorectal cancer, hormone sensitive or hormone refractory prostate cancer, colorectal cancer, ovarian cancer, hepatocellular cancer, renal cell cancer, soft tissue sarcoma and small cell lung cancer. In some preferred embodiments the cancer to be treated is ER/PR−, HER2+ breast cancer, triple negative (negative for expression of progesterone receptor, estrogen receptor and human epidermal growth factor receptor-2) breast cancer, ER and/or PR+HER2+ breast cancer, NSCLC (squamous and/or nonsquamous) or gastro-esophageal junction (GEJ) cancer.

In one aspect, the subject to be treated with the combination is a human with a cancer that is refractory to (has progressed on) treatment with one or more chemotherapeutic agents and/or refractory to treatment with one or more antibodies. The checkpoint inhibitor and oncolytic virus combination of the invention may be administered to a human with cancer identified as a candidate for checkpoint inhibitor therapy. In some embodiments, the oncolytic virus is administered to potentiate the effects of checkpoint inhibitor therapy and is administered prior to administering the checkpoint inhibitor.

In some aspects, treatment is determined by a clinical outcome such as, without limitation, increase, enhancement or prolongation of anti-tumor activity by T cells, an increase in the number of anti-tumor T cells or activated T cells as compared with the number prior to treatment or a combination thereof. In another aspect, clinical outcome is tumor stabilization, tumor regression, tumor shrinkage, and/or increase in overall survival.

In a further aspect, the method further comprises administering a chemotherapeutic agent, targeted therapy, radiation, cryotherapy, or hyperthermia therapy to the subject prior to simultaneously with or after treatment with the combination therapy.

Related embodiments of the present invention provide a pharmaceutical combination for use in the treatment of cancer or for use in the manufacture of a medicament for treating cancer, in a mammal wherein the combination comprises an oncolytic virus, preferably an oncolytic rhabdovirus, and a checkpoint inhibitor. In some embodiments, the pharmaceutical combination comprises a human or humanized monoclonal antibody against CTLA4 or PD-1/PD-L1 and a VSV or Maraba strain rhabdovirus optionally modified to increase selectivity for cancer cells such as, without limitation, VSVdelta51 or Maraba MG1.

In a further aspect, a kit for use in inducing an immune response in a mammal is provided including an oncolytic virus, preferably an oncolytic rhabodvirus and a checkpoint inhibitor. In some embodiments, the kit comprises a VSV or Maraba strain rhabdovirus optionally modified to increase selectivity for cancer cells such as, without limitation, VSVdelta51 or Maraba MG1 that expresses MAGEA3, a Human Papilloma Virus E6/E7 fusion protein, human Six-Transmembrane Epithelial Antigen of the Prostate Protein, Cancer Testis Antigen 1 or a variant thereof and a checkpoint inhibitor, preferably a PD-1, PD-L1 and/or CTLA-4 checkpoint inhibitor and optionally may further comprise a second virus that is immunologically distinct from the oncolytic rhadovirus so that it may act as the “prime” in a heterologous prime-boost vaccination and which expresses the same antigen as the oncolytic rhabdovirus. The kit may further comprise instructions for using the combination for treating cancer.

Other embodiments of the invention are discussed throughout this application. Any embodiment discussed with respect to one aspect of the invention applies to other aspects of the invention as well, and vice versa. The embodiments in the Detailed Description and Example sections are understood to be non-limiting embodiments of the invention that are applicable to all aspects of the invention.

The terms “inhibiting,” “reducing,” or “preventing,” or any variation of these terms, when used in the claims and/or the specification includes any measurable decrease or complete inhibition to achieve a desired result. Desired results include but are not limited to palliation, reduction, slowing, or eradication of a cancerous or hyperproliferative condition, as well as an improved quality or extension of life.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “mammal” refers to humans as well as non-human mammals.

A “checkpoint inhibitor” as used herein means an agent which acts on surface proteins which are members of either the TNF receptor or B7 superfamilies, including agents which bind to negative co-stimulatory molecules including without limitation CTLA-4, PD-1, TIM-3, BTLA, VISTA, LAG-3, and/or their respective ligands, including PD-L1.

The terms “Programmed Death 1”, “Programmed Cell Death 1”, “Protein PD-1” “PD-1” and “PD1” are used interchangeably, and include variants, isoforms, species homologs of human PD-1, and analogs having at least one common epitope with PD-1. The complete PD-1 sequence can be found under GenBank Accession No. U64863.

The terms “cytotoxic T lymphocyte-associated antigen-4,” “CTLA-4,” “CTLA4,” and “CTLA-4 antigen” are used interchangeably, and include variants, isoforms, species homologs of human CTLA-4, and analogs having at least one common epitope with CTLA-4. The complete CTLA-4 nucleic acid sequence can be found under GenBank Accession No. L15006.

It is to be understood that “combination therapy” envisages the simultaneous, sequential or separate administration of the components of the combination. In one aspect of the invention, “combination therapy” envisages simultaneous administration of the oncolytic virus and checkpoint inhibitor. In a further aspect of the invention, “combination therapy” envisages sequential administration of the oncolytic virus and checkpoint inhibitor. In another aspect of the invention, “combination therapy” envisages separate administration of the oncolytic virus and checkpoint inhibitor. Where the administration of the oncolytic virus and checkpoint inhibitor is sequential or separate, the oncolytic virus and checkpoint inhibitor are administered within time intervals that allow that the therapeutic agents show a cooperative e.g., synergistic, effect. In preferred embodiments, the oncolytic virus and checkpoint inhibitor are administered within 1, 2, 3, 6, 12, 24, 48, 72 hours, or within 4, 5, 6 or 7 days or within 8, 9, 10, 11, 12, 13, 14, 15, 16, 17, 18, 19, 20, 21, 22, 23, 24, 25, 26, 27, 28, 29, 30 or 31 days of each other. In some embodiments, a first dose of the oncolytic virus is administered (i.e. treatment with the oncolytic virus is initiated) prior to a first dose of the checkpoint inhibitor (i.e. prior to initiating treatment with the checkpoint inhibitor) or vice versa and may include a phase where treatment with the oncolytic virus and treatment with the checkpoint inhibitor overlap. In other embodiments, a first dose of the oncolytic virus may be administered on or about the same time as a first dose of the checkpoint inhibitor. In other embodiments, a first dose of oncolytic virus is administered after a first dose (or second, third or subsequent dose) of checkpoint inhibitor and may include a phase where treatment with the oncolytic virus and treatment with the checkpoint inhibitor overlap.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Treatment schema for co-administration of a checkpoint inhibitor (aCTLA4; anti-CTLA4 antibody) and an oncolytic rhabdovirus (MG1 GFP; Maraba double mutant expressing green fluorescent protein (GFP)) to mice carrying subcutaneous CT26 tumors. Group 1 (Control) received PBS; Group 2 (MG1/GFP) received 3 intravenous injections of MG1 GFP only on days 1, 3 and 5; Group 3 (MG1/GFP+CTLA4) received 3 intravenous injections of MG1 GFP on days 1, 3, and 5 and 8 intraperitoneal injections of anti-CTLA4 antibody on days 1, 4, 7, 10, 13, 16, 19 and 22; Group 4 (CTLA4) received 8 intraperitoneal injections of anti-CTLA4 antibody alone on days 1, 4, 7, 10, 13, 16, 19 and 22. Immune analysis was performed on day 10.

FIG. 2. CT26-specific immune response on day 10—total IFN-γ response. The percentage of CD8+ T cells secreting IFN-γ after ex vivo exposure to AH1, the immunodominant CT26 epitope (gp70₄₂₃₋₄₃₁) is shown for each Group. Co-administration of MG1/GFP and CTLA4 increased the percentage of CD8 T cells secreting IFN-γ in response to AH1.

FIG. 3. CT26-specific immune response on day 10—IFN-γ single positive T cells. The percentage of CD8+ T cells secreting IFN-γ (but not TNFα) after ex vivo exposure to AH1, the immunodominant CT26 epitope (gp70₄₂₃₋₄₃₁) is shown for each Group. Co-administration of MG1/GFP and CTLA4 increased the percentage of IFN-γ single positive CD8+ T cells in response to AH1.

FIG. 4. CT26-specific immune response on day 10—IFN-γ/TNFα double positive T cells. The percentage of CD8+ T cells secreting IFN-γ and TNFα after ex vivo exposure to AH1, the immunodominant CT26 epitope (gp70₄₂₃₋₄₃₁) is shown for each Group. Co-administration of MG1/GFP and CTLA4 increased the percentage of IFN-γ/TNFα double positive CD8+ T cells in response to AH1.

FIG. 5. Tumor growth curve. The tumor volume of mice from each treatment Group over time beginning at Day 0 is depicted.

FIG. 6. Kaplan-Meier survival curve. The percent survival of mice from each treatment Group over time beginning at Day 0 is depicted.

FIG. 7. Treatment schema for co-administration of a checkpoint inhibitor (anti-PD-1 antibody) and an oncolytic rhabdovirus expressing the hDCT tumor antigen (MG1 hDCT) following a priming administration with adenovirus expressing the hDCT tumor antigen (Ad-hDCT); to mice carrying metastatic lung tumors. Group 1 (Control) received PBS; Group 2 (αPD-1) received 11 intraperitoneal injections of anti-PD-1 antibody only on days 8, 10, 13, 15, 17, 20, 22, 24, 27, 29 and 31; Group 3 (Ad:MG1 hDCT) received a single administration of 2×10⁸ pfu of AdhDCT on day 5 followed by 2 intravenous injections of MG1 hDCT on days 14 and 17; Group 4 (Ad:MG1 hDCT+αPD-1) received a single administration of 2×10⁸ pfu of AdhDCT on day 5 followed by (i) 2 intravenous injections of MG1 hDCT on days 14 and 17 and (ii) 11 intraperitoneal injections of anti-PD-1 antibody only on days 8, 10, 13, 15, 17, 20, 22, 24, 27, 29 and 31. Immune analyses were performed on Days 14, 20 and 27.

FIGS. 8A-8F. Immune analysis at peak prime timepoint (Day 14). FIGS. 8A and 8B illustrate the percentage of lymphocytes staining positive for CD8 and CD4 markers in PBMCs from each treatment Group at Day 14. FIG. 8C illustrates the percentage of CD8+ T cells secreting IFN-γ (in total). FIGS. 8D-8F illustrate the percentage of CD8+ T cells secreting IFN-γ only (FIG. 8D), IFN-γ and TNFα (FIG. 8E) and IFN-γ, TNFα and IL-2 (FIG. 8F) from each treatment Group after ex vivo exposure to SVY, the immunodominant epitope of DCT (DCT₁₈₀₋₁₈₈) at Day 14.

FIGS. 9A-9D. Immune Analysis at Peak Boost (Day 20). FIGS. 9A-9B illustrate the percentage of lymphocytes staining positive for CD8 markers in PBMCs from each treatment Group (FIG. 9A) and the number of CD8+ T cells in blood from each treatment Group (FIG. 9B) at Day 20. FIGS. 9C-9D illustrate the percentage of CD8+ T cells secreting IFN-γ in total and the number of CD8+ T cells secreting IFN-γ in total per μl from each treatment Group in response to SVY at Day 20.

FIGS. 10A-F. Phenotype analysis of SVY-specific T cells at peak boost (Day 20). FIGS. 10A-10C illustrate the percentage of CD8+ T cells secreting IFN-γ only (i.e. excluding those that also secrete TNFα and/or IL-2) (FIG. 10A), IFN-γ and TNFα (FIG. 10B) and IFN-γ, TNFα and IL-2 (FIG. 10C) from each treatment Group after ex vivo exposure to SVY. FIGS. 10D-10F illustrate the number of CD8+ T cells secreting IFN-γ only (FIG. 10D), IFN-γ and TNFα (FIG. 10E) and IFN-γ, TNFα and IL-2 (FIG. 10F) per μ1 of blood from each treatment Group after ex vivo exposure to SVY.

FIGS. 11A-11D. Immune Analysis—Late Boost (Day 27). FIGS. 11A-11B compare the percentage of lymphocytes staining positive for CD8 markers (FIG. 11A) and the number of CD8+ T cells in blood (FIG. 11B) in the MG1-hDCT treatment Group (“Prime:Boost”) and the combination treatment Group (MG1-hDCT+anti-PD-1 antibody; “Prime:boost PD1”) at Day 27. FIGS. 11C-11D compares the percentage of CD8+ T cells secreting IFN-γ in total and the number of CD8+ T cells secreting IFN-γ in total per μ1 in blood from these treatment Groups in response to SVY at Day 27.

FIGS. 12A-12F. Phenotype analysis of SVY specific T cells at late boost (Day 27). FIGS. 12A-12C illustrate the percentage of CD8+ T cells secreting IFN-γ only (i.e. excluding those that also secrete TNFα and/or IL-2) (FIG. 12A), IFN-γ and TNFα (FIG. 12B) and IFN-γ, TNFα and IL-2 (FIG. 12C) from the specified treatment Groups after ex vivo exposure to SVY. FIGS. 12D-12F illustrate the number of CD8+ T cells secreting IFN-γ only (FIG. 12D), IFN-γ and TNFα (FIG. 12E) and IFN-γ, TNFα and IL-2 (FIG. 12F) per μl of blood from the specified treatment Groups after ex vivo exposure to SVY.

FIG. 13. Kaplan-Meier Survival Curve. The percent survival of mice from each treatment Group over time beginning at Day 0 is depicted

FIGS. 14A-C. Graphs illustrating the effect of anti PD-1 antibody administered as a single dose at the same time as a priming administration of hDCT (“Ab day 7 (concomitant)”) (FIG. 14A), as a single dose 3 days after priming administration of hDCT (“Ab day 10 (sequential)”) (FIG. 14B) and as multiple doses starting 3 days after priming administration of hDCT (“Ab continuous (starting day 10)”) (FIG. 14C) on mouse weight compared to prime-boost alone (“No Ab”).

FIG. 15 Graph illustrating the effect of anti PD-1 antibody treatment, initiated on the same day as priming administration of hDCT (“Ab day 7 (concomitant)”), on Maraba virus titers compared to prime-boost treatment alone (“No Ab”).

FIGS. 16A-16B FIG. 16A: Microarray analysis of 4T1 cells infected for 24h at an MOI of 3 with MG1-GFP or irradiated MG1-GFP. The heat map includes the top genes that were enriched more than 4-fold as compared to uninfected cells. FIG. 16B: Microarray analysis of EMT6 cells infected for 24h at an MOI of 3 with MG1-GFP or irradiated MG1-GFP. The heat map includes the top genes that were enriched more than 4-fold as compared to uninfected cells.

FIGS. 17A-17B FIG. 17A: Flow cytometry analysis of surface PDL1 expression of 4T1 cells after a 24h incubation in virus-cleared, MG1-infected 4T1 conditioned media. FIG. 17B: 4T1-tumor bearing mice were treated IT for 5 consecutive days with MG1-GFP. The graphs show the percentage of the T cells that were Tregs in the spleens (left panel) and tumors (right panel) 12 days after the last virus injection. Two-tailed unpaired T-test: **: p<0.01.

FIGS. 18A-18B FIG. 18A: 4T1-tumor bearing mice were treated IT for 5 consecutive days with MG1-GFP followed by a combination of anti-CTLA4 and anti-PD1 (100m each) injected IP, every second day, for a total of 5 injections. The tumors were collected and measured. Each tumor volume was divided by the average tumor volume of the control animals for each experiment (4 experiments are included on the graph). Statistical analysis using unpaired two-tailed t-test: *: p<0.05, **: p<0.01 ***: p<0.001. FIG. 18B: Tumor growth (left panel) and Kaplan-Meier survival analysis (right panel) of 4T1 tumor bearing mice using the tumor re-challenge model where the first tumors were left untreated (NT) or treated with MG1-GFP IT and the second tumors were treated or not with the ICIs (100 ug each, IP) for a total of 5 injections, every second day, starting on day 25. The dashed lines represent the days of MG1 treatment. Statistical analysis for tumor measurements: *: p<0.05, **: p<0.01 ***: p<0.001 (unpaired multiple two-tailed t-test). Difference between NT and MG1+ICI groups are indicated by *, differences between MG1 and MG1+ICI groups are indicated by # and differences between ICI and MG1+ICI groups are indicated by x. For survival curves: **: p<0.01 ***: p<0.001 (Mantel-Cox test).

FIG. 19 Schematic of treatment arms in a Phase I/PhaseII clinical trial examining the effects of a prime:boost strategy employing adenovirus vaccine (AdMA3) and MG1 (MG1MAE3), each with transgenic MAGE-A3 insertion in patients with incurable MAGE-A3-expressing solid tumors. Arm B and C begin AdMA3 dosing on day (−14).

FIG. 20 Graph showing the fold change in PDL1 expression (post-treatment vs. pre-treatment) in individual tumor biopsies from patients of the clinical trial of FIG. 19 treated with AdMA3 (“Ad”), MG1MA3 (“MG1”), or both at the indicated dose.

FIG. 21 Graph showing the fold change in PDL1 expression (post-treatment vs. pre-treatment) from pooled tumor biopsies for all doses in Arms A, B and C in patients of the current clinical trial.

### DETAILED DESCRIPTION OF THE INVENTION

It has been found that combination therapy with an oncolytic virus (e.g. oncolytic rhabdovirus) and a checkpoint inhibitor results in unexpected improvement in the treatment of cancer. When administered simultaneously, sequentially or separately, the oncolytic virus and the checkpoint inhibitor interact cooperatively and even synergistically to significantly improve survival relative to single administration of either component with no apparent adverse effects or reduction in virus titer. This unexpected effect may allow a reduction in the effective dose of each component, leading to a reduction in side effects and enhancement of clinical effectiveness of the compounds and treatment.

In several embodiments, a combination therapy for use in the treatment and/or prevention of cancer and/or the establishment of metastases in a mammal is provided comprising co-administering to the mammal (i) a replication competent oncolytic virus in combination with (ii) an immune checkpoint inhibitor. In preferred embodiments, the replication competent oncolytic virus is administered prior to the immune checkpoint inhibitor.

Oncolytic Virus

In preferred embodiments, the replication competent oncolytic virus of the combination is an oncolytic rhabdovirus.

Oncolytic rhabdoviruses have several advantages as the oncolytic virus for use in the combination including the following: (1) Antibodies to the oncolytic rhabdoviruses will be rare to non-existent in most populations of the world. (2) rhabdoviruses replicate more quickly than other oncolytic viruses such as adenovirus, reovirus, measles, parvovirus, retrovirus, and HSV. (3) Rhabdovirus grow to high titers and are filterable through 0.2 micron filter. (4) The oncolytic rhabdoviruses and recombinants thereof have a broad host range, capable of infecting many different types of cancer cells and are not limited by receptors on a particular cell (e.g., coxsackie, measles, adenovirus). (5) The rhabdovirus of the invention is amenable to genetic manipulation. (6) The rhabdovirus also has a cytoplasmic life cycle and do not integrate in the genetic material a host cell, which imparts a more favorable safety profile.

The archetypal rhabdoviruses are rabies and vesicular stomatitis virus (VSV), the most studied of this virus family. Rhabdovirus is a family of bullet shaped viruses having non-segmented (−)sense RNA genomes. The family Rhabdovirus includes, but is not limited to: Arajas virus, Chandipura virus (AF128868/gi:4583436, AJ810083/gi:57833891, AY871800/gi:62861470, AY871799/gi:62861468, AY871798/gi:62861466, AY871797/gi:62861464, AY871796/gi:62861462, AY871795/gi:62861460, AY871794/gi:62861459, AY871793/gi:62861457, AY871792/gi:62861455, AY871791/gi:62861453), Cocal virus (AF045556/gi:2865658), Isfahan virus (AJ810084/gi:57834038), Maraba virus (SEQ ID ON: 1-6 of U.S. Pat. No. 8,481,023, incorporated herein by reference; HQ660076.1), Carajas virus (SEQ ID NO:7-12 of U.S. Pat. No. 8,481,023, incorporated herein by reference, AY335185/gi:33578037), Piry virus (D26175/gi:442480, Z15093/gi:61405), Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus (DQ457103/gi|91984805), Perinet virus (AY854652/gi:71842381), Tupaia virus (NC_007020/gi:66508427), Farmington, Bahia Grande virus (SEQ ID NO:13-18 of U.S. Pat. No. 8,481,023, incorporated herein by reference, KM205018.1), Muir Springs virus (KM204990.1), Reed Ranch virus, Hart Park virus, Flanders virus (AF523199/gi:25140635, AF523197/gi:25140634, AF523196/gi:25140633, AF523195/gi:25140632, AF523194/gi:25140631, AH012179/gi:25140630), Kamese virus, Mosqueiro virus, Mossuril virus, Barur virus, Fukuoka virus (AY854651/gi:71842379), Kern Canyon virus, Nkolbisson virus, Le Dantec virus (AY854650/gi:71842377), Keuraliba virus, Connecticut virus, New Minto virus, Sawgrass virus, Chaco virus, Sena Madureira virus, Timbo virus, Almpiwar virus (AY854645/gi:71842367), Aruac virus, Bangoran virus, Bimbo virus, Bivens Arm virus, Blue crab virus, Charleville virus, Coastal Plains virus, DakArK 7292 virus, Entamoeba virus, Garba virus, Gossas virus, Humpty Doo virus (AY854643/gi:71842363), Joinjakaka virus, Kannamangalam virus, Kolongo virus (DQ457100/gi|91984799 nucleoprotein (N) mRNA, partial cds); Koolpinyah virus, Kotonkon virus (DQ457099/gi|91984797, AY854638/gi:71842354); Landjia virus, Manitoba virus, Marco virus, Nasoule virus, Navarro virus, Ngaingan virus (AY854649/gi:71842375), Oak-Vale virus (AY854670/gi:71842417), Obodhiang virus (DQ457098/gi|91984795), Oita virus (AB116386/gi:46020027), Ouango virus, Parry Creek virus (AY854647/gi:71842371), Rio Grande cichlid virus, Sandjimba virus (DQ457102/gi|91984803), Sigma virus (AH004209/gi:1680545, AH004208/gi:1680544, AH004206/gi:1680542), Sripur virus, Sweetwater Branch virus, Tibrogargan virus (AY854646/gi:71842369), Xiburema virus, Yata virus, Rhode Island, Adelaide River virus (U10363/gi:600151, AF234998/gi:10443747, AF234534/gi:9971785, AY854635/gi:71842348), Berrimah virus (AY854636/gi:71842350]), Kimberley virus (AY854637/gi:71842352), or Bovine ephemeral fever virus (NC_002526/gi:10086561).

In a preferred embodiment, the oncolytic virus of the combination is a wild type Maraba strain rhabdovirus or a variant thereof that has optionally been genetically modified e.g. to enhance tumor selectivity. The Maraba virus may be e.g. a Maraba virus containing a substitution at amino acid 242 of the G protein and/or at amino acid 123 of the M protein as described at col. 2, lines 24-42 of U.S. Pat. No. 9,045,729, the entire contents of which are incorporated herein by reference. In a particularly preferred embodiment, the Maraba virus is Maraba MG1 as described in Brun et al., Mol. Ther., 18(8):1440-1449 (2010). Maraba MG1 is a genetically modified Maraba strain rhabdovirus containing a G protein mutation (Q242R) and an M protein mutation (L123W) that renders the virus hypervirulent in cancer cells yet attenuated in normal cells.

In another preferred embodiment, the oncolytic rhadovirus is a VSV strain or a variant thereof that has optionally been genetically modified e.g. to enhance tumor selectivity. In a particularly preferred embodiment, the VSV comprises a deletion of methionine at position 51 of the M protein as described in Stojdl et al., Cancer Cell., 4(4):263-75 (2003), the contents of which are incorporated herein by reference.

In other preferred embodiments, the oncolytic rhabdovirus expresses one or more tumor associated antigens such as oncofetal antigens such as alphafetoprotein (AFP) and carcinoembryonic antigen (CEA), surface glycoproteins such as CA 125, oncogenes such as Her2, melanoma-associated antigens such as dopachrome tautomerase (DCT), GP100 and MART 1, cancer-testes antigens such as the MAGE proteins and NY-ESO1, viral oncogenes such as HPV E6 and E7, and proteins ectopically expressed in tumours that are usually restricted to embryonic or extraembryonic tissues such as PLAC or a variant of a tumor-associated antigen. In such case, the combination therapy is preferably administered to a human with a cancer expressing the tumor associated antigen. A “variant” of a tumor associated antigen refers to a protein that (a) includes at least one tumor associated antigenic epitope from the tumor associated antigenic protein and (b) is at least 70%, preferably at least 80%, more preferably at least 90% or at least 95% identical to the tumor associated antigenic protein. A database summarizing well accepted antigenic epitopes is provided by Van der Bruggen P, Stroobant V, Vigneron N, Van den Eynde B in “Database of T cell-defined human tumor antigens: the 2013 update.” Cancer Immun 2013 13:15 and www.cancerimmunity.org/peptide. Thus, in various embodiments, the oncolytic rhabdovirus (e.g. VSVdelta51 or Maraba MG1) of the combination encodes a protein comprising an amino acid sequence of SEQ ID NO: 1, SEQ ID NO: 4, SEQ ID NO: 7, SEQ ID NO: 10, SEQ ID NO: 13 or a variant at least 95% identical thereto. In related embodiments, the oncolytic rhabdovirus of the combination includes a reverse complement and RNA version of a transgene comprising a nucleotide sequence of SEQ ID NO: 2, 3, 5, 6, 8, 9, 11, 12, or 14.

In particularly preferred embodiments, the oncolytic rhadovirus expresses MAGEA3, Human Papilloma Virus E6/E7 fusion protein, human Six-Transmembrane Epithelial Antigen of the Prostate protein, or Cancer Testis Antigen 1. Oncolytic rhabdovirus expressing each of these tumor-associated antigens has been demonstrated to increase survival in relevant animal cancer models in a prime-boost strategy (WIPO publication no. WO 2014/127478). “Prime-boost” as used herein means administering (preferably intravascularly) to a mammal with cancer an (replicative) oncolytic rhabodvirus expressing a natural tumor-associated antigen associated with that cancer and to which the mammal has a pre-existing immunity to boost a pre-existing immunity, wherein the pre-existing immunity in the mammal is preferably established by a priming administration of the tumor-associated antigen to the mammal prior to administering the oncolytic rhabdovirus. Preferably, the mammal has a cancer in which expression of the tumor-associated antigen has been detected/identified.

The priming step may be accomplished by administering (using any suitable administration route including but limited to intravenous, intramuscular or intranasal administration) the tumor-associated antigen per se or, preferably, by administering the tumor-associated antigen via a vector such as an adenoviral, poxviral (e.g. vaccinia virus), retroviral (e.g. lentivirus) or alpha virus (e.g. semliki forest) vector, or a plasmid or loaded antigen-presenting cell such as a dendritic cell. The vector used to administer the priming administration with tumor-associated antigen is immunologically distinct from (i.e. is heterologous to) the oncolytic virus expressing tumor-associated antigen administered to boost immunity in the mammal (e.g. in the case where the oncolytic virus expressing tumor-associated antigen is an oncolytic rhabdovirus, the priming vector is either not a rhabdovirus or is an immunologically distinct rhabdovirus). Generally, the vector is modified to express the antigen using well-established recombinant technology and is administered in an amount effective to generate an immune response in the mammal. By way of example, intramuscular administration of at least about 10⁷ pfu of adenoviral vector expressing a tumor-associated antigen to a mouse is sufficient to generate an immune response. For treatment of humans, for example, about 10⁸-10¹², 10⁹-10¹¹ or 10¹⁰ pfu of adenovral vector expressing a tumor-associated antigen may be administered to generate a priming immune response.

Once an immune response has been generated in the mammal by a priming administration of the tumor-associated antigen (e.g. via adenovirus vector), the oncolytic rhabdovirus expressing the same tumor-associated antigen in an amount effective for oncolytic viral therapy is administered at least once within a suitable immune response interval which may be for example, at least about 24 hours, preferably at least about 2-4 days or longer, e.g. within about one week, within about two weeks, within about three weeks or within about four weeks.

In some embodiments, a first boosting administration of oncolytic rhabdovirus expressing a tumor-associated antigen occurs about two weeks after a single priming administration of the same tumor-associated antigen (e.g. via adenovirus vector) which may be followed by a second boosting administration about 15-20 days, about 16-19 days or about 17 days after the single priming administration. In related embodiments, a first dose of the checkpoint inhibitor is administered after a single priming administration and prior to a first boosting administration of the oncolytic rhabdovirus expressing the same tumor-associated antigen and preferably includes a treatment phase wherein administration of the checkpoint inhibitor and administration of the oncolytic rhabdovirus expressing the same tumor-associated antigen overlap. In other embodiments, a second dose of the checkpoint inhibitor is administered after a first, second (and optionally third, fourth, fifth and so on) boosting administration. In related embodiments, the checkpoint inhibitor is administered weekly, every other week or every three weeks.

The MAGE family of genes encoding tumor specific antigens is discussed in De Plaen et al., Immunogenetics 40:360-369 (1994). MAGEA3 is expressed in a wide variety of tumours including melanoma, non-small cell lung cancer, head and neck cancer, colorectal cancer and bladder cancer. Tumor associated antigenic epitopes have been already identified for MAGEA3. Accordingly, a variant of the MAGEA3 protein may be, for example, an antigenic protein that includes at least one tumor associated antigenic epitope selected from the group consisting of: EVDPIGHLY (SEQ ID NO: 1), FLWGPRALV (SEQ ID NO: 2), KVAELVHFL (SEQ ID NO: 3), TFPDLESEF (SEQ ID NO:4), VAELVHFLL (SEQ ID NO: 5), MEVDPIGHLY (SEQ ID NO: 6), EVDPIGHLY (SEQ ID NO: 7), REPVTKAEML (SEQ ID NO: 8), AELVHFLLL (SEQ ID NO: 9), MEVDPIGHLY (SEQ ID NO: 10), WQYFFPVIF (SEQ ID NO: 11), EGDCAPEEK (SEQ ID NO: 12), KKLLTQHFVQENYLEY (SEQ ID NO: 13), RKVAELVHFLLLKYR (SEQ ID NO: 14), KKLLTQHFVQENYLEY (SEQ ID NO: 15), ACYEFLWGPRALVETS (SEQ ID NO: 16), RKVAELVHFLLLKYR (SEQ ID NO: 17), VIFSKASSSLQL (SEQ ID NO: 18), VIFSKASSSLQL (SEQ ID NO: 19), VFGIELMEVDPIGHL (SEQ ID NO: 20), GDNQIMPKAGLLIIV (SEQ ID NO: 21), TSYVKVLHHMVKISG (SEQ ID NO: 22), RKVAELVHFLLLKYRA (SEQ ID NO: 23), and FLLLKYRAREPVTKAE (SEQ ID NO: 24); and that is at least 70%, 80%, 90%, or 95% identical to the MAGEA3 protein. It may be desirable for variants of a tumor associated antigenic protein to include only antigenic epitopes that have high allelic frequencies, such as frequencies greater than 40% of the population. Accordingly, preferred examples of variants of MAGEA3 may include proteins that include at least one antigenic epitope selected from the group consisting of: FLWGPRALV (SEQ ID NO: 25), KVAELVHFL (SEQ ID NO: 26), EGDCAPEEK (SEQ ID NO: 27), KKLLTQHFVQENYLEY (SEQ ID NO: 28), RKVAELVHFLLLKYR (SEQ ID NO: 29), and KKLLTQHFVQENYLEY (SEQ ID NO: 30); and that is at least 70%, 80%, 90% or 95% identical to the MAGE A3 protein.

Human Papilloma Virus (HPV) oncoproteins E6/E7 are constitutively expressed in cervical cancer (Zur Hausen, H (1996) Biochem Biophys Acta 1288:F55-F78). Furthermore, HPV types 16 and 18 are the cause of 75% of cervical cancer (Walboomers J M (1999) J Pathol 189: 12-19). An oncolytic rhabdovirus expressing a fusion protein of the E6/E7 oncoproteins of HPV types 16 and 18, which was mutated to remove oncogenic potential, has been shown to increase the number and percentage of antigen-specific CD8+ T cells in a heterologous prime:boost setting.

Six-Transmembrane Epithelial Antigen of the Prostate (huSTEAP) is a recently identified protein shown to be overexpressed in prostate cancer and up-regulated in multiple cancer cell lines, including pancreas, colon, breast, testicular, cervical, bladder, ovarian, acute lyphocytic leukemia and Ewing sarcoma (Hubert R S et al., (1999) Proc Natl Acad Sci 96: 14523-14528). The STEAP gene encodes a protein with six potential membrane-spanning regions flanked by hydrophilic amino- and carboxyl-terminal domains. An oncolytic rhabdovirus expressing huSTEAP has been shown to increase the number and percentage of antigen-specific CD8+ T cells in a heterologous prime:boost setting.

Cancer Testis Antigen 1 (NYES01) is a cancer/testis antigen expressed in normal adult tissues, such as testis and ovary, and in various cancers (Nicholaou T et al., (2006) Immunol Cell Biol 84:303-317). Cancer testis antigens are a unique family of antigens, which have restricted expression to testicular germ cells in a normal adult but are aberrantly expressed on a variety of solid tumours, including soft tissue sarcomas, melanoma and epithelial cancers. An oncolytic rhabdovirus expressing NYES01 has been shown to increase the number and percentage of antigen-specific CD8+ T cells in a heterologous prime:boost setting.

In other embodiments, an oncolytic rhabdovirus expressing a tumor-associated antigen is co-administered with a checkpoint inhibitor to a mammal with cancer, wherein the mammal has a naturally existing immunity to the tumor-associated antigen.

Thus, in several embodiments, a method for treating and/or preventing cancer in a mammal is provided comprising co-administering to a mammal with cancer (i) an oncolytic rhabdovirus expressing a natural tumor associated antigen naturally associated with the cancer and to which the mammal has a pre-existing immunity and (ii) a checkpoint inhibitor, whereby the pre-existing immunity in the mammal is preferably established by administering the tumor antigen to the mammal prior to administering the oncolytic rhabdovirus. In preferred embodiments, the oncolytic rhabdovirus is intravascularly administered to the mammal. In other preferred embodiments, the pre-existing immunity in the mammal is established by administering a viral vector (e.g. adenovirus) expressing the tumor-associated antigen to the mammal prior to administering the oncolytic rhabdovirus.

Routes of administration of the oncolytic virus of the combination will vary, naturally, with the location and nature of the lesion, and include, e.g., intradermal, transdermal, parenteral, intravascular (intravenous or intra-arterial), intramuscular, intranasal, subcutaneous, regional, percutaneous, intratracheal, intraperitoneal, intravesical, intratumoral, inhalation, perfusion, lavage, direct injection, alimentary, and oral administration and formulation. In preferred embodiments, a pharmaceutical composition comprising the oncolytic virus (e.g. oncolytic rhabdovirus) of the combination and a pharmaceutically acceptable carrier is administered to a mammal with cancer by intratumoral injection and/or is administered intravascularly, although the pharmaceutical composition may alternatively be administered intratumorally, parenterally, intravenously, intrarterially, intradermally, intramuscularly, transdermally or even intraperitoneally as described in U.S. Pat. Nos. 5,543,158, 5,641,515 and 5,399,363 (each specifically incorporated herein by reference in its entirety). As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 1, 2, 3, 4, 5, 6 or more dose application over a 1, 2, 3, 4, 5, 6-week period or more. A two-week regimen may be repeated one, two, three, four, five, six or more times. During a course of treatment, the need to complete the planned dosings may be re-evaluated.

The treatments may include various “unit doses.” Unit dose is defined as containing a predetermined quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) or viral particles for viral constructs. Unit doses range from 10³, 10⁴, 10⁵, 10⁶, 10⁷, 10⁸, 10⁹, 10¹⁰, 10¹¹, 10¹², 10¹³ pfu or vp and higher. Alternatively, depending on the kind of virus and the titer attainable, one will deliver 1 to 100, 10 to 50, 100-1000, or up to about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles (vp) to the patient or to the patient's cells.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

Checkpoint Inhibitor

Immune checkpoints regulate T cell function in the immune system. T cells play a central role in cell-mediated immunity. Checkpoint proteins interact with specific ligands which send a signal into the T cell and switch off or inhibit T cell function. Cancer cells in turn exploit this by driving high level expression of checkpoint proteins on their surface resulting in control of the T cell expressing checkpoint proteins on the surface of T cells that enter the tumor microenvironment, thus suppressing the anti-cancer immune response.

An immune checkpoint inhibitor for use in the combination is any compound inhibiting the function of an immune checkpoint protein. Inhibition includes reduction of function and full blockade. In particular the immune checkpoint protein is a human immune checkpoint protein. Thus the immune checkpoint inhibitor preferably is an inhibitor of a human immune checkpoint protein. Immune checkpoint proteins are described in the art (see e.g. Pardoll, Nature Rev. Cancer 12(4): 252-264 (2012).

Checkpoint proteins include, without limitation CTLA4, PD-1 and its ligands PD-L1 and PD-L2, B7-H3, B7-H4, HVEM, TIM3, GALS, LAG3, VISTA, KIR, TIGIT, and BTLA. The pathways involving LAG-3, BTLA, B7H3, B7H4, TIM3, and KIR are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489).

Preferred immune checkpoint protein inhibitors are antibodies, preferably human or humanized monoclonal antibodies, that specifically recognize immune checkpoint proteins. A number of CTLA-4, PD1, PDL-1, PD-L2, LAG-3, BTLA, B7H3, B7H4, TIM3, TIGIT and KIR inhibitors have been described.

CTLA-4 checkpoint inhibitors include, without limitation, ipilimumab (a fully human CTLA-4 blocking antibody presently marketed under the name Yervoy® (Bristol-Myers Squibb)), tremelimumab (referenced in Ribas et al., J. Clin. Oncol. 31:616-622 (2013)), antibodies disclosed in U.S. Patent Application Publication Nos. 2005/0201994, 2002/0039581, and 2002/086014, the contents of each of which are incorporated herein by reference, and antibodies disclosed in U.S. Pat. Nos. 5,811,097, 5,855,887, 6,051,227, 6,984,720, 6,682,736, 6,207,156, 5,977,318, 6,682,736, 7,109,003 and 7,132,281, the contents of each of which are incorporated herein by reference.

PD-1 inhibitors include without limitation humanized antibodies blocking human PD-1 such as lambrolizumab (e.g. disclosed as hPD109A and its humanized derivatives h409A11, h409A16 and h409A17 in U.S. Pat. No. 8,354,509, incorporated herein by reference; and in Hamid et al., N. Engl. J. Med. 369: 134-144 (2013)), pidilizumab (CT-011; disclosed in Rosenblatt et al., J Immunother. 34:409-418 (2011)), as well as fully human antibodies such as nivolumab (CAS Registry Number: 946414-94-4; previously known as MDX-1106 or BMS-936558, Topalian et al., N. Eng. J. Med. 366:2443-2454 (2012), disclosed in U.S. Pat. No. 8,008,449, incorporated herein by reference) or an antibody comprising the heavy and light chain variable regions of any of these antibodies. Pidilizumab is a fully human IgG4 monoclonal antibody that has shown efficacy for treatment of diffuse large B-cell lymphoma in human clinical trials. Nivolumab is a fully human IgG4 monoclonal antibody that has shown efficacy for treatment of advanced treatment-refractory malignancies (e.g. melanoma, renal cell carcinoma, and NSCLC). Other PD-1 inhibitors may include fusion proteins such as the PD-L2-Fc fusion protein also known as B7-DC-Ig or AMP-244 (disclosed in Mkrtichyan M, et al. J Immunol. 189:2338-47 2012). AMP224 is undergoing phase I testing as a monotherapy in treatment of subjects with advanced cancer.

In a preferred embodiment, the immune checkpoint inhibitor is nivolumab or an isolated anti-PD-1 antibody comprising a heavy chain variable region comprising the heavy chain variable region amino acid sequence of nivolumab and/or a light chain variable region comprising the light chain variable region amino acid sequence of nivolumab. The heavy chain sequence of nivolumab is:

(SEQ ID NO: 31)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK

The light chain sequence of nivolumab is:

(SEQ ID NO: 32)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEMRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

In some preferred embodiments, the checkpoint inhibitor comprises a heavy chain and/or a light chain sequence at least 85%, at least 90%, at least 91%, at least 92%, at least 93%, at least 94%, at least 95%, at least 96%, at least 98%, at least 99% or 100% to the heavy chain and/or light chain sequence of nivolumab.

Immune checkpoint inhibitors also include, without limitation, humanized or fully human antibodies blocking PD-L1 such as pembrolizumab (CAS Registry Number 1374853-91-4; also known as MK-3475) (disclosed in WO2009/114335), MEDI-4736 (disclosed in U.S. Pat. No. 8,779,108, incorporated herein by reference), MPDL33280A (disclosed in U.S. Pat. No. 8,217,149, the contents of which are incorporated herein by reference), MIH1 (Affymetrix obtainable via eBioscience (16.5983.82)), BMS-936559 and MSB0010718C (Avelumab) or an antibody comprising the heavy and light chain variable regions of any of these antibodies. BMS-936559 is a fully human IgG4 monoclonal antibody demonstrated to show efficacy in treatment of melanoma, NSCLC, renal cell carcinoma and ovarian cancer in human clinical trials (administered bi-weekly). Pembrolizumab is a humanized IgG4 monoclonal antibody with a stabilizing SER228PRO sequence alteration in the Fc region undergoing clinical trials for treatment of progressive, locally advanced or metastatic carcinoma, melanoma or NSCLC, which binds to PD-1 and prevents the interaction of PD-1 with its ligands PD-L1 and PD-L2. MPDL33280A is a monoclonal antibody undergoing testing in combination with the BRAF inhibitor vemurafenib in subjects with BRAF V600-mutant metastatic melanoma and in combination with bevacizumab which targets VEGFR in subjects with advanced solid tumors. MEDI-4736 is in phase I clinical testing in patients with advanced malignant melanoma, renal cell carcinoma, NSCLC and colorectal cancer.

In a particularly preferred embodiment, the immune checkpoint inhibitor is pembrolizumab or an isolated anti-PD-1 antibody comprising a heavy chain variable region comprising the heavy chain variable region amino acid sequence of pembrolizumab and/or a light chain variable region comprising the light chain variable region amino acid sequence of pembrolizumab. The heavy chain sequence of pembrolizumab is:

(SEQ ID NO: 33)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK

The light chain sequence of pembrolizumab is:

(SEQ ID NO: 34)
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

In some preferred embodiments, the checkpoint inhibitor comprises a heavy chain and/or a light chain sequence at least 85%, at least 90%, at least 91%, at least 92%, at least 93%, at least 94%, at least 95%, at least 96%, at least 98%, at least 99% or 100% to the heavy chain and/or light chain sequence of pembrolizumab.

In preferred embodiments, an immune checkpoint inhibitor of the combination is selected from a CTLA-4, PD-1 or PD-L1 inhibitor, such as, without limitation, pembrolizumab, ipilimumab, tremelimumab, labrolizumab, nivolumab, pidilizumab, AMP-244, MEDI-4736, MPDL33280A, or MIH1. Known inhibitors of these immune checkpoint proteins may be used as such or analogues may be used, in particular chimerized, humanized or human forms of antibodies.

As the skilled person will know, alternative and/or equivalent names may be in use for certain antibodies mentioned above. Such alternative and/or equivalent names are interchangeable in the context of the present invention. For example it is known that lambrolizumab is also known under the alternative and equivalent names MK-3475 and pembrolizumab.

Other immune checkpoint inhibitors of the combination include, without limitation, agents targeting immune checkpoint proteins and pathways involving PD-L2, LAG3, BTLA, B7H4, TIM3 and TIGIT. For example, human PD-L2 inhibitors known in the art include MIH18 (described in Pfistershammer et al., Eur J Immunol. 36:1104-1113 (2006)). LAG3 inhibitors known in the art include soluble LAG3 (IMP321, or LAG3-Ig disclosed in U.S. Patent Application Publication No. 2011-0008331, incorporated herein by reference, and in Brignon et al., Clin. Cancer Res. 15:6225-6231 (2009)) as well as mouse or humanized antibodies blocking human LAG3 (for instance IMP701 and others described U.S. Patent Application Publication No. 2010-0233183, incorporated herein by reference), or fully human antibodies blocking human LAG3 (such as BMS-986016 and the antibodies disclosed in U.S. Patent Application Publication No. 2011-0150892, incorporated herein by reference).

BTLA inhibitors of the combination, include without limitation antibodies blocking human BTLA interaction with its ligand (such as 4C7 disclosed in U.S. Pat. No. 8,563,694, incorporated herein by reference).

B7H4 checkpoint inhibitors include, without limitation, antibodies to human B7H4 (disclosed in WO 2013025779 A1, and in U.S. Patent Application Publication No. 2014/0294861, incorporated herein by reference) or soluble recombinant forms of B7H4 (such as disclosed in U.S. Patent Application Publication No. 2012/0177645, incorporated herein by reference, or Anti-human B7H4 clone H74: eBiocience #14-5948).

B7-H3 checkpoint inhibitors, include, without limitation, antibodies neutralizing human B7-H3 (e.g. MGA271 disclosed as BRCA84D and derivatives in U.S. Patent Application Publication No. 2012/0294796, incorporated herein by reference).

TIM3 checkpoint inhibitors include, without limitation, antibodies targeting human TIM3 (e.g. as disclosed in U.S. Pat. No. 8,841,418, incorporated herein by reference, or the anti-human TIM3, blocking antibody F38-2E2 disclosed by Jones et al., J Exp Med., 205(12):2763-79 (2008)). KIR checkpoint inhibitors include, without limitation, Lirilumab (described in Romagne et al., Blood, 114(13):2667-2677 (2009)) Known inhibitors of immune checkpoint proteins may be used in their known form or analogues may be used, in particular chimerized forms of antibodies, most preferably humanized forms. TIGIT checkpoint inhibitors preferably inhibit interaction of TIGIT with polovirus receptor (CD155) and include, without limitation, antibodies targeting human TIGIT, such as those disclosed in U.S. Pat. No. 9,499,596 and U.S. Patent Application Publication Nos. 20160355589, 20160176963 and polovirus variants such as those disclosed in U.S. Pat. No. 9,327,014.

In some aspects, the combination described herein includes (i) more than one immune checkpoint inhibitor and (ii) an oncolytic virus within the various aspects of the invention. Preferably, the more than one immune checkpoint inhibitor is selected from a CTLA-4, a PD-1 or a PD-L1 inhibitor. For example concurrent therapy of ipilimumab (anti-CTLA4) with Nivolumab (anti-PD1) has demonstrated clinical activity that appears to be distinct from that obtained in monotherapy (Wolchok et al., N. Eng. J. Med., 369:122-33 (2013)). Other examples include a LAG3 checkpoint inhibitor and an anti-PD-1 checkpoint inhibitor (Woo et al., Cancer Res. 72:917-27 (2012)) or a LAG3 checkpoint inhibitor and a PD-L1 checkpoint inhibitor (Butler et al., Nat. Immunol., 13:188-195 (2011)).

In other aspects, the combination described herein includes (i) one or more checkpoint inhibitors and one or more additional therapeutic agents that have been shown to improve the efficacy of the one or more checkpoint inhibitors and (ii) an oncolytic virus. For example, Lirilumab (also known as anti-KIR, BMS-986015 or IPH2102, as disclosed in U.S. Pat. No. 8,119,775 in combination with ipilimumab (clinicaltrials.gov NCT01750580) or in combination with nivolumab (clinicaltrials.gov NCT01714739). Another example is an agent targeting ICOS and a CTLA-4 checkpoint inhibitor (Fu et al., Cancer Res., 71:5445-54 (2011), or an agent targeting 4-1BB (e.g. urelumab) and a CTLA-4 checkpoint inhibitor (Curran et al., PloS 6(4):9499 (2011)). Other examples include PD-1/PD-L1 checkpoint inhibitors and pazopanib, sunitinib, dasatinib, INCR024360, PegIFN-2b, Tarceva, Cobimetinib, and/or Trametinib, Debrafinib. In some preferred embodiments, the combination comprises an oncolytic rhabdovirus and (i) Nivolumab+Pazopanib/Sunitinib/Ipilumamb, (ii) Nivolumab+Dasatinib, (iii) Pembrolizumab+INCR024360 (iv) Pembrolizumab+pazopanib (v) Pembrolizumab+PegIFN-2b (vi) MED14736+Dabrafenib/Trametinib (vii) MPDL3280A+Tarceva or (viii) MPDL3280A+Cobimetinib.

The checkpoint inhibitor as disclosed herein can be administered by various routes including, for example, orally or parenterally, such as intravenously, intramuscularly, subcutaneously, intraorbitally, intracapsularly, intraperitoneally, intrarectally, intracisternally, intratumorally, intravasally, intradermally or by passive or facilitated absorption through the skin using, for example, a skin patch or transdermal iontophoresis, respectively. The checkpoint inhibitor also can be administered to the site of a pathologic condition, for example, intravenously or intra-arterially into a blood vessel supplying a tumor.

The total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the composition to treat a pathologic condition in a subject depends on many factors including the age and general health of the subject as well as the route of administration and the number of treatments to be administered. In view of these factors, the skilled artisan would adjust the particular dose as necessary. In general, the formulation of the composition and the routes and frequency of administration are determined, initially, using Phase I and Phase II clinical trials.

In certain embodiments, the checkpoint inhibitor is administered in 0.01-0.05 mg/kg, 0.05-0.1 mg/kg, 0.1-0.2 mg/kg, 0.2-0.3 mg/kg, 0.3-0.5 mg/kg, 0.5-0.7 mg/kg, 0.7-1 mg/kg, 1-2 mg/kg, 2-3 mg/kg, 3-4 mg/kg, 4-5 mg/kg, 5-6 mg/kg, 6-7 mg/kg, 7-8 mg/kg, 8-9 mg/kg, 9-10 mg/kg, at least 10 mg/kg, or any combination thereof doses. In certain embodiments the checkpoint inhibitor is administered at least once a week, at least twice a week, at least three times a week, at least once every two weeks, at least once every three weeks, or at least once every month or multiple months. In related embodiments, the checkpoint inhibitor is administered once per week, once every other week, once every three weeks or once every month. In certain embodiments, the checkpoint inhibitor is administered as a single dose, in two doses, in three doses, in four doses, in five doses, or in 6 or more doses. In a preferred embodiment, the checkpoint inhibitor is pembrolizumab and is administered at a schedule of 2 mg/kg (preferably as an intravenous infusion over 30 minutes) once every 3 weeks.

### EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Oncolytic Rhabdovirus+Checkpoint Inhibitor

The effects of co-administering a checkpoint inhibitor and an oncolytic rhabdovirus were assessed in a clinically relevant immunocompetent syngeneic tumor model.

### Materials and Methods

BALB/c mice were engrafted with 5×10⁵ CT26 (colon carcinoma) cells subcutaneously. Tumors were allowed to grow until they reached approximately 250 mm³. Mice were randomized to one of 4 groups (Table 1) ensuring equal mean tumour and variances:

TABLE 1

| Group | Treatment | Number |
|---|---|---|
| 1 | Control | 5 |
| 2 | MG1/GFP | 5 |
| 3 | MG1/GFP + | 9 |
| | CTLA4 | |
| 4 | CTLA4 | 5 |

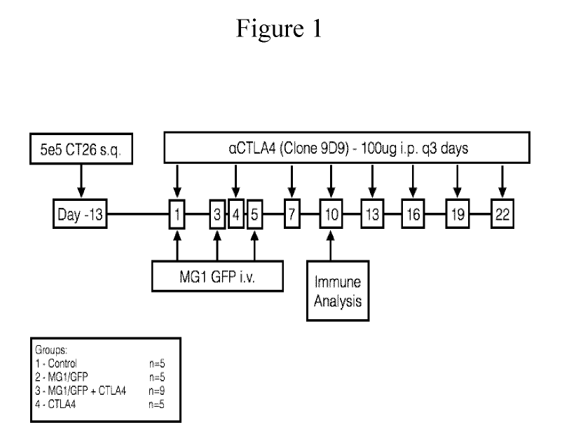

MG1/GFP, a genetically modified Maraba strain rhabdovirus containing a G protein mutation (Q242R) and an M protein mutation (L123W) and expressing the heterologous protein GFP (green fluorescent protein) was administered at a dose of 2×10⁸ plaque forming units (PFUs) intravenously on days 1 and 3 and 5×10⁸ PFU intravenously on day 5. Mouse-derived anti-CTLA4 monoclonal antibody (Clone 9D9; BioXCell Cat. No. BE0164) was administered by intraperitoneal injection at a dose of 100 μg every three days. The co-administration regimen is depicted at FIG. 1. Tumor measurements were recorded 3 days a week by caliper measurement. Tumor volumes were calculated using the following formula: 4/3*π*L/2*(W/2)²−where L=length and W=width. Survival was recorded for all mice. Mice were considered at endpoint once tumours were greater than 1500 mm³.

Immune analyses were performed on Day 10 following the first dose of MG1/GFP. Immune analyses were completed on peripheral blood mononuclear cells (PBMCs) by ex vivo peptide re-stimulation and were stained for a panel of cytokines to assess the quantity of CT26 AH1-specific T cells as well as determining poly-functionality. Polyfunctionality was assessed by quantifying IFN-γ single positive and IFN-γ/TNF-α double positive. Antibodies for flow cytometry were from BD Biosciences: IFNγ-APC Cat#554413; TNFα-FITC Cat #554418; CD107a-PE Cat#558661 or from eBiosciences: CD8-Alexa700 Cat#56-0081-82; CD4-PerCp-Cy5.5 Cat#45-0042-82. Peptides for restimulation were from Biomer Technology: CT26 AH1-SPSYVYHQF; VSV/MG1 N-MPYLIDFGL. Briefly, CT26-specific T cell responses were measured on Day 10. Peripheral blood mononucleated cells were incubated in complete RPMI with CT26 AH1 peptide for CT26-specific CD8+ T-cell (re-)stimulation. Incubation was performed in incubator (37 C, 5% CO₂, 95% humidity) for 5 hours and 40 minutes, with brefeldin A (1 μg/ml) during the last 4 hours. Cells were treated with antibodies targeting CD16/CD32 before staining with fluorescent-labeled antibodies targeting T-cell surface markers. Then, cells were permeabilized and fixed and stained for intracellular cytokines. Data were acquired using a FACSCanto flow cytometer.

### Results

Anti-Tumor Responses.

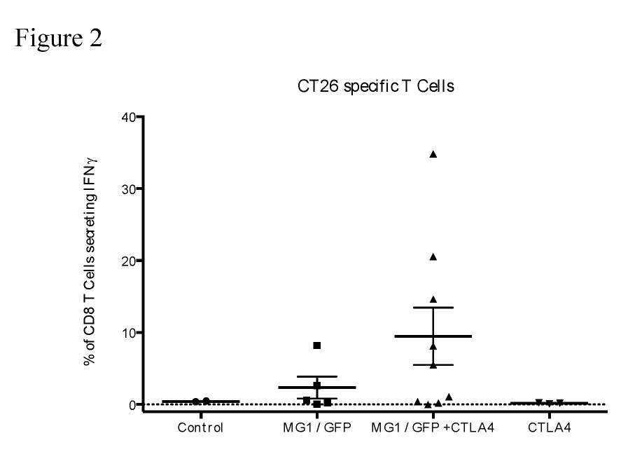

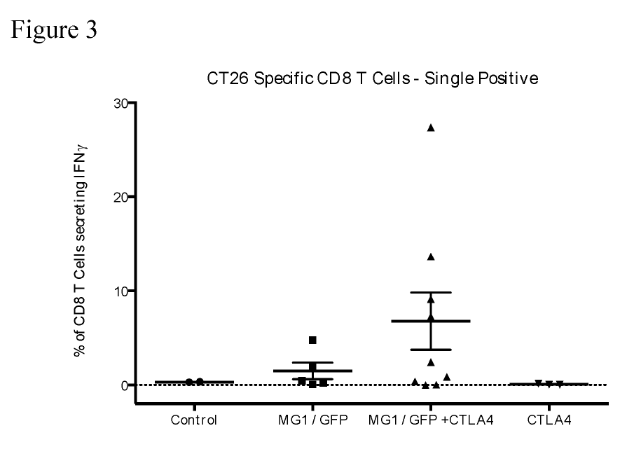

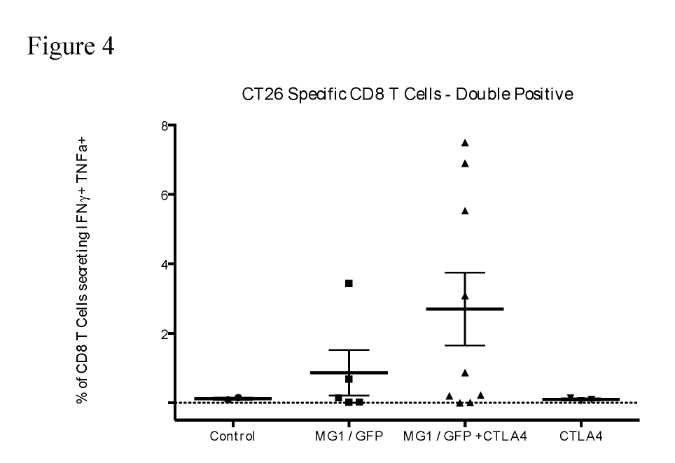

Co-administration of anti-CTLA4 antibody with MG1/GFP led to an increased anti-CT26 immune response. FIG. 2 illustrates the percentage of CD8+ T cells expressing IFN-γ in total in response to CT26 antigen for mice in each of the four Groups. FIGS. 3 and 4 illustrate the percentage of CD8+ T cells secreting only IFN-γ (single positive, excluding cells that also express TNF-α) and secreting IFN-γ and TNFα (double positive, excluding cells that only express IFN-γ) respectively in response to CT26 antigen. FIGS. 2-4 demonstrate that co-administering a checkpoint inhibitor with an oncolytic rhabdovirus increases the percentage of CD8+ T cells specific for the immunodominant CT26 antigen.

Tumor Size.

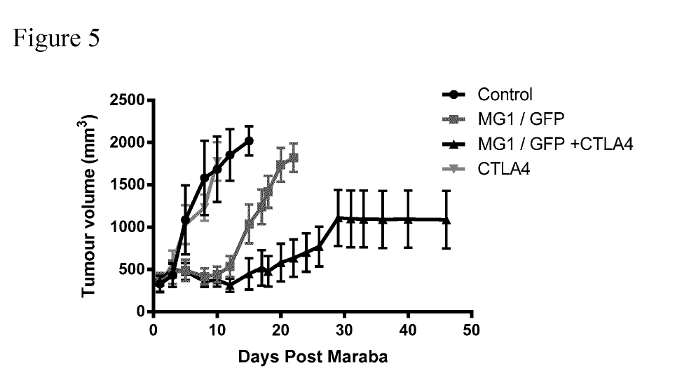

Tumors in control animals (Control, FIG. 5) reached a mean size of 2,000 mm³ by Day 15. Treatment with anti-CTLA4 antibody alone did not slow tumor growth (CLTA4, FIG. 5). Treatment with MG1/GFP alone slowed tumor growth, although by Day 22, tumors in all mice reached a mean size of 1800 mm³ (MG1/GFP, FIG. 5). Treatment with a combination of MG1/GFP and CTLA4 inhibitor was statistically superior to control, anti-CTLA-4 and MG-1/GFP alone in terms of tumor growth and tumors in animals treated with the combination of MG1/GFP and CTLA4 did not exceed 1500 mm³ throughout the evaluation period (MG1/GFP+CTLA4, FIG. 5).

Survival Analysis.

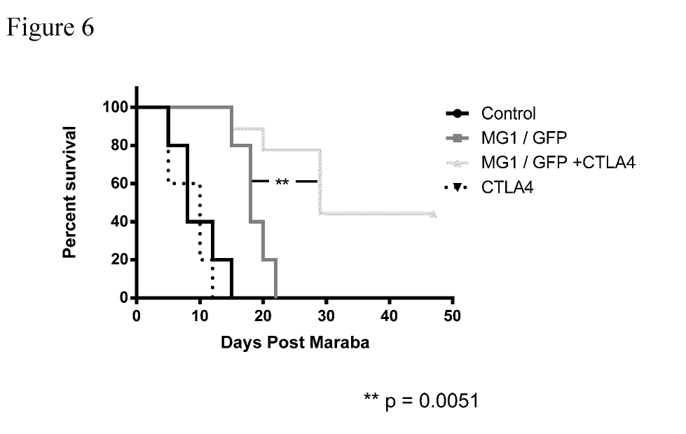

Survival of animals from each treatment Group was analyzed. The data are presented in FIG. 6 as Kaplan-Meier Curves. The regimen of MG1/GFP in combination with anti-CTLA4 antibody was statistically superior to treatment with either agent alone or control (Log-rank Mantel-Cox test; p values 0.0051 combination compared to MG1/GFP alone). Median survival times were 8 days (control), 10 days (anti-CTLA4 alone), 18 days (MG1/GFP alone) and 29 days (combination). Four of the nine mice in the combination treatment Group were alive at day 47, the end of the study. In contrast, none of the mice in the Group administered MG1/GFP alone survived past Day 22.

Combination treatment with a checkpoint inhibitor—anti-CTLA-4—and an oncolytic rhabdovirus—MG1/GFP, significantly delayed tumor growth compared to either treatment alone and a significant survival benefit was observed with the combination treatment compared to either agent alone.

### Example 2

### Checkpoint Inhibitor+Oncolytic Rhabdovirus Prime-Boost

The impact of co-administering a checkpoint inhibitor—anti-PD-1 antibody—and a Maraba rhabodvirus expressing a tumor antigen (following a priming administration with the same tumor antigen, as described in Pol et al., Mol Ther 22(2):420-429 (2014), the entire contents of which are incorporated herein by reference) on the anti-tumor immune response was assessed in a clinically relevant syngeneic B16 lung metastasis model.

Material and Methods.

C57BL/6 mice were engrafted with 2.5×10⁵ B16F10 mouse melanoma cells intravenously and tumors were allowed to seed for 5 days. Mice were assigned to one of 4 groups (Table 2)

TABLE 2

| Group | Group name | Drug | Treatment (Days) | Number |
|---|---|---|---|---|
| 1 | Control | Control | No treatment | 5 |
| 2 | Anti-PD-1 | Anti-PD-1 | D8, 10, 13, 15, 17, | 5 |
| | | | 20, 22, 24, 27, 29, 31 | |
| 3 | Prime/boost | Ad-hDCT: | Ad hDCT: D5 | 10 |
| | | MG1 | MG1 hDCT: D14, 17 | |
| | | hDCT | | |
| 4 | Combination | Ad-hDCT: | Ad hDCT: D5 | 9 |
| | | MG1 | MG1 hDCT: D14, 17 | (evaluable) |
| | | hDCT + | Anti-PD-1: D8, 10, | |
| | | anti-PD-1 | 13, 15, 17, 20, 22, 24, | |
| | | | 27, 29, 31, 34, 36, 38 | |

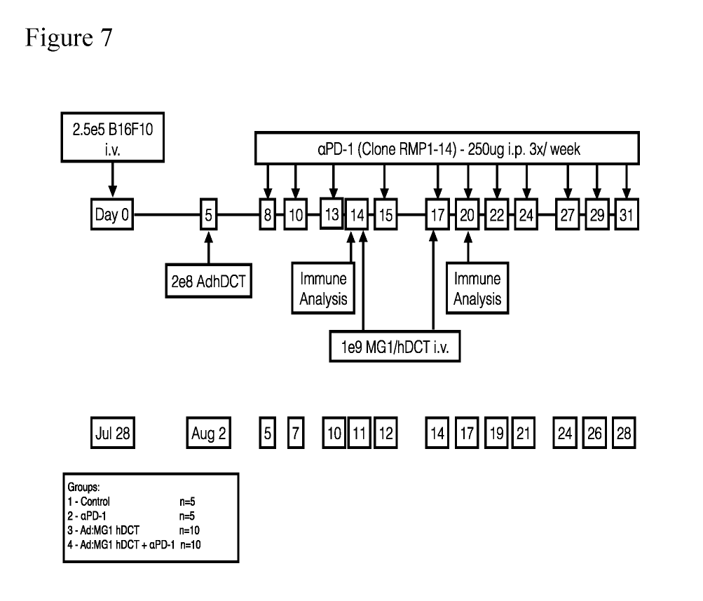

Ad-hDCT, a replication-deficient adenovirus (E1/E3-deletion) based on human serotype 5 engineered to express the human dopachrome tautomerase (hDCT) transgene, was administered at a dose of 2×10⁸ pfu intramuscularly. MG1-hDCT, the MG1 Maraba virus engineered to express the hDCT transgene, was administered intravenously at a dose of 1×10⁹ pfu. Anti-PD-1 antibody (BioXCell Cat. No. BE0146) was administered by intraperitoneal injection at a dose of 250 μg 3 days a week for 5 weeks. A graphical overview of the treatment schema is at FIG. 7.
Immune analyses were performed on Day 14 (following prime) and Day 20 (anticipated peak boost) and Day 27. Immune analyses were completed on PBMCs by ex vivo peptide re-stimulation and were stained for a panel of cytokines to assess the quantity of DCT-specific T cells as well as determining poly-functionality. Polyfunctionality was assessed by quantifying IFN-γ single positive, IFN-γ/TNF-α double positive, and IFN-γ/TNF-α/IL-2 triple positive cells. CD107a marker staining detects cytolytic activity of CD8+ T cells by measuring degranulation, a prerequisite for cytolysis. Antibodies for flow cytometry were from BD Biosciences: IFN-γ-APC Cat#554413; TNFα-FITC Cat#554418; IL-2-BV421 Cat#562969; CD107a-PE Cat#558661 or from eBiosciences: CD8-Alexa700 Cat#56-0081-82; CD4-PerCp-CY5.5 Cat#45-0042-82. Peripheral blood mononucleated cells were incubated in complete RPMI with SVY peptide (corresponding to the immunodominant epitope of DCT (DCT₁₈₀₋₁₈₈) that binds to H-2K^{b}; 2 μg/ml) for DCT-specific CD8+ T-cell (re-)stimulation. Incubation was performed in incubator (37 C, 5% CO₂, 95% humidity) for 5 hours and 40 minutes, with brefeldin A (1 μg/ml) during the last 4 hours. Cells were treated with antibodies targeting CD16/CD32 before staining with fluorescent-labeled antibodies targeting T-cell surface markers. Then, cells were permeabilized and fixed and stained for intracellular cytokines. Data were acquired using a FACSCanto flow cytometer

Survival was recorded for all mice. Mice were considered at endpoint if exhibiting severe respiratory distress.

Results.

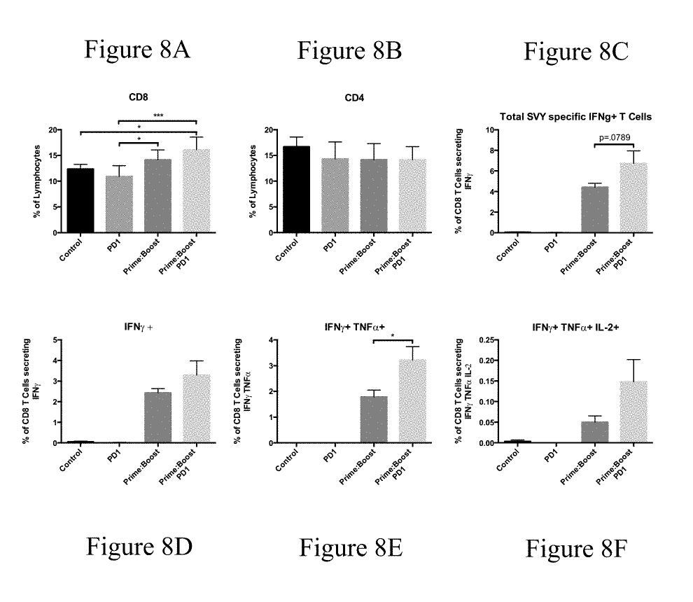

Intracellular cytokine staining (ICS) following 5 hours and 40 minutes of peptide stimulations of peripheral blood (staining with antibodies recognizing IFN-γ, TNF-α and IL-2) at the peak prime timepoint (Day 14) revealed an increase in the percentage of CD8+ T cells staining for the following cytokine(s): IFN-γ (single positive), IFN-γ+TNF-α (double positive) and IFN-γ+TNF-α+IL-2 (triple positive) for the combination treatment group versus either treatment alone. The results are illustrated at FIGS. 8A-F. As can be seen from FIGS. 8A-8B, combination treatment with checkpoint inhibitor and oncolytic rhabdovirus resulted in an increase in the percentage of CD8+ T cells compared to the other treatment Groups. Treatment with checkpoint inhibitor alone did not affect the total global percentage of CD8+ T cells expressing IFN-γ (including those that also express TNF-α and/or IL-2), or the percentage of CD8+ T cells expressing IFN-γ only (excluding cells that also express TNF-α and/or IL-2) or expressing IFN-γ and TNFα or expressing IFN-γ, TNF-α and IL-2 (FIGS. 8C-8F; compare lanes “PD1” to “control” lanes). Combination treatment with oncolytic rhabdovirus expressing a tumor antigen and a checkpoint inhibitor (following a priming administration of the same tumor antigen) significantly increased the total global percentage of CD8+ T cells expressing IFN-γ (FIG. 8C),the percentage of single positive (IFN-γ) CD8+ T cells (FIG. 8D), the percentage of double positive (IFN-γ+TNFα) CD8+ T cells (FIG. 8E) and the percentage of triple positive (IFN-γ+TNFα+IL-2) CD8+ T cells (FIG. 8F) compared to treatment with oncolytic rhabodvirus expressing the tumor antigen alone (FIGS. 8C-8F; compare lanes “Prime:Boost PD1” to lanes “Prime:Boost”).

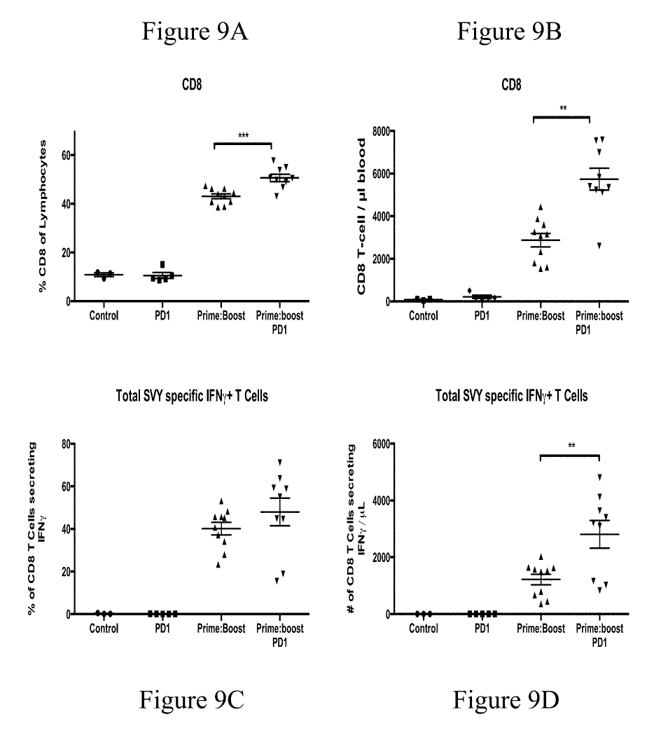

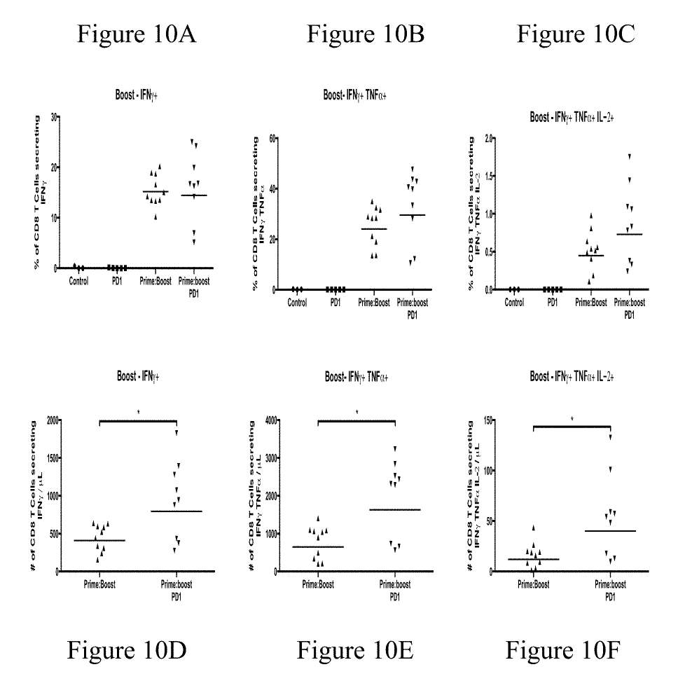

ICS staining using the same conditions for peripheral blood collected at the peak boost time point (Day 20) demonstrated a statistically significant increase in CD8+ T cell frequency and number in blood in the combination treatment group (“Prime:boost PD1”) relative to single treatment groups (“PD1” or “Prime:Boost”). See FIGS. 9A-9B. At the same time point, there was a significant increase in the total number of DCT-specific IFN-γ-producing CD8+ T cells upon combination treatment vs prime/boost or anti-PD-1 treatment alone (FIG. 9D). The addition of PD-1 also led to significant increases of higher quality DCT specific T cells, both IFN-γ/TNFα double positive (FIG. 10B) and IFN-γ/TNF-α/IL-2 triple positive cells (FIG. 10C). There was no difference in DCT-specific T cells when assessing CD8 frequency (FIG. 9A); however, the increased expansion of the CD8+ T cell pool in the PD-1 combination group is what led to significantly increased numbers of DCT-reactive CD8+ T cells.

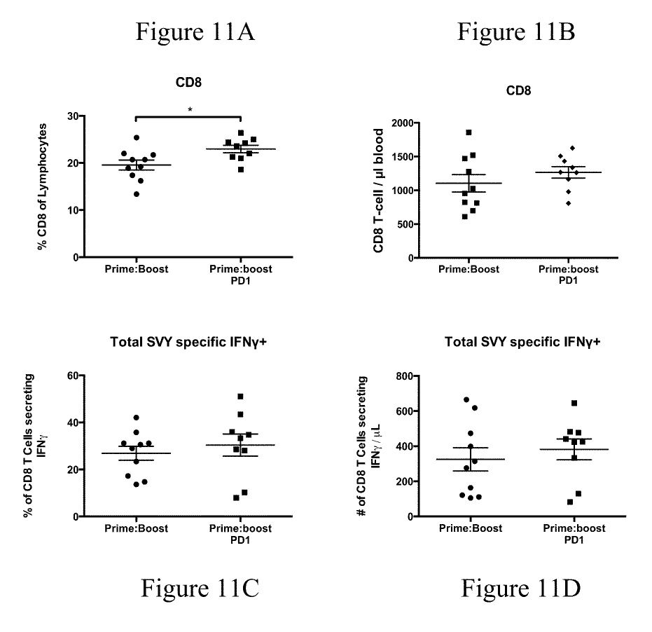

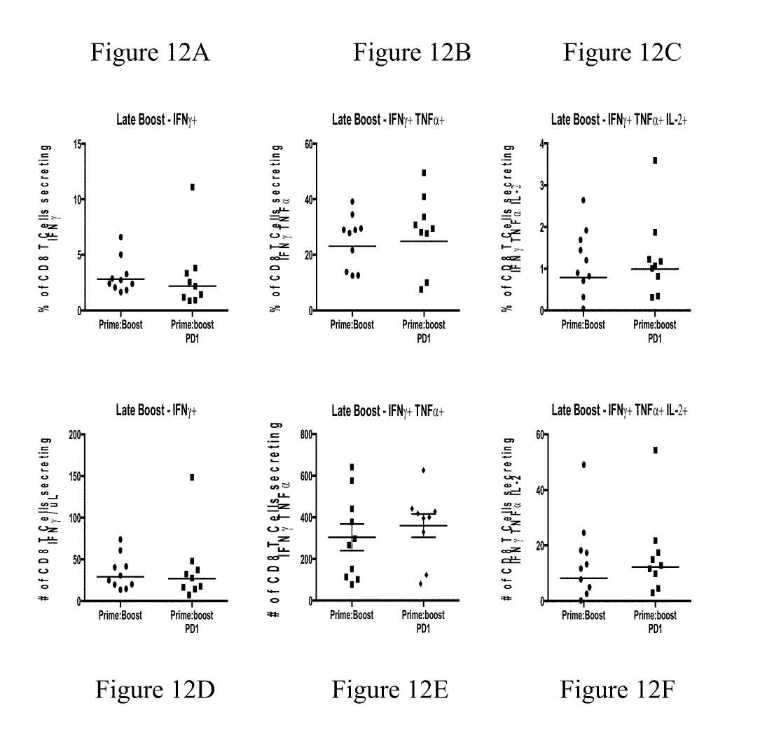

ICS staining using the same conditions for peripheral blood collected at the later boost time point (Day 27 of the study) demonstrated an increase in the frequency of CD8+ T cells in blood in the combination group when compared to the prime/boost group (FIG. 11A) but not in the number of CD8+ T cells (FIG. 11B). No difference in IFN-γ producing T cells was noted at this time point (FIGS. 11C-11D). There was no statistically significant difference in the frequency or number of single, double and triple positive CD8+ T cells between any of the groups at this time point (FIGS. 12A-F).

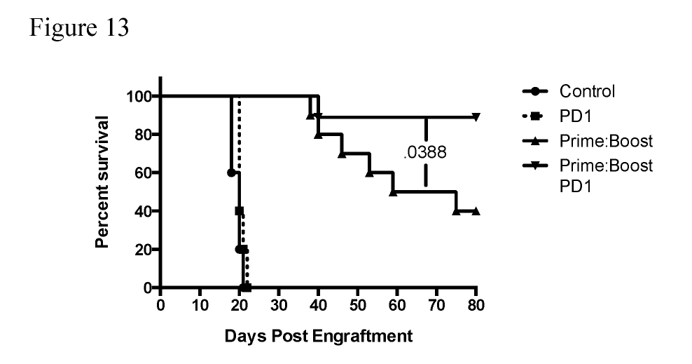

Analysis of subject survival was performed. The data is shown at FIG. 13 as Kaplan-Meier Curves. The regimen of Ad-hDCT:MG1 hDCT in combination with anti-PD-1 antibody was statistically significantly superior to treatment with either agent alone or control (Log-rank Mantel-Cox test, p values 0.0388 combination compared to prime/boost alone). Median survival times were 20 days (“Control”), 20 days (anti-PD-1 alone (“PD1”)) and 67 days (“Prime/Boost”). By study end (Day 80), 8 of 9 animals in the combination group (“Prime:Boost PD1”) had not reached endpoint, so no median survival time was calculated for this group.

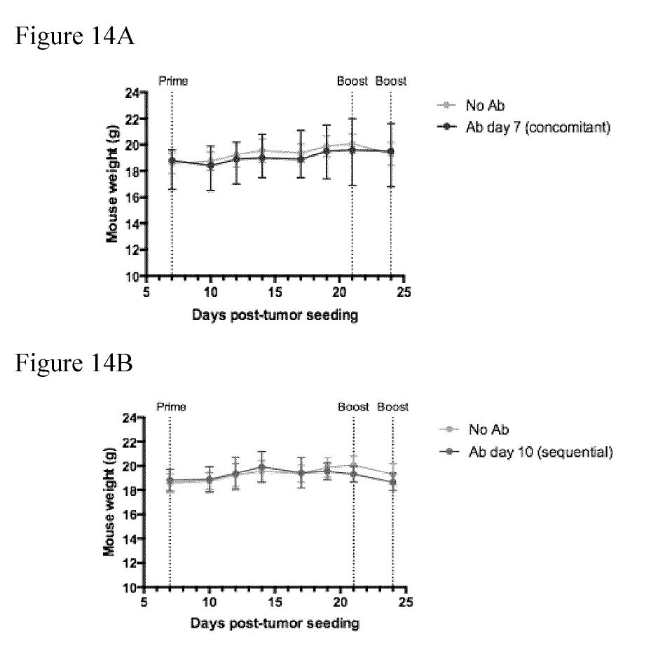

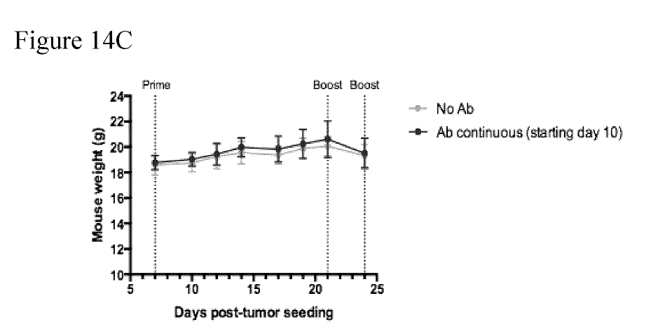

The effect of combination therapy with anti-PD-1 and MG1 Maraba rhabdovirus expressing hDCT following a priming administration of hDCT (prime-boost) on mouse weight was assessed compared to prime-boost alone. As can be seen from FIGS. 14A-14C, administering anti-PD-1 antibody did not impact the weight of mice relative to prime-boost alone regardless of whether the antibody was given as a single dose at the same time as the prime (ad-hDCT administration) (FIG. 14A), as a single dose 3 days after prime (FIG. 14B) or given continuously as multiple doses starting 3 days after the prime (FIG. 14C). Thus, the toxicity of combination therapy is not greater than prime-boost alone regardless of administration regimen.

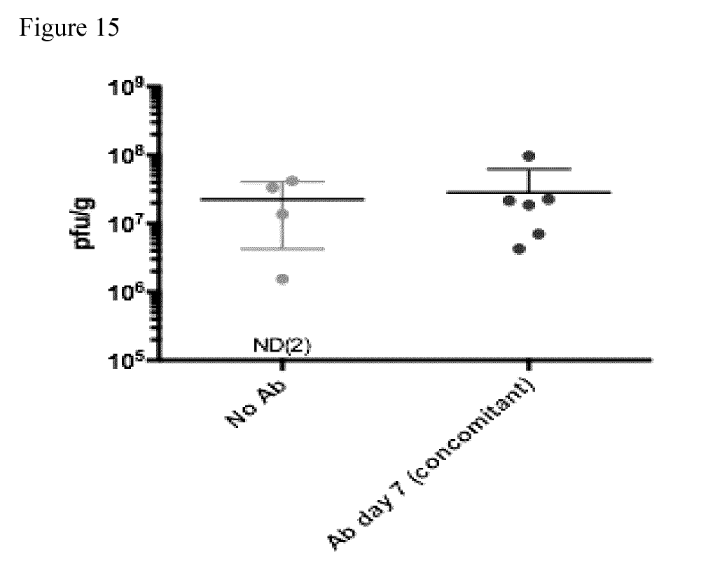

The effect of combination therapy with anti-PD-1 and MG1 Maraba rhabdovirus expressing hDCT following a priming administration of hDCT (prime-boost) on Maraba virus titer was assessed compared to prime-boost alone. As can be seen from FIG. 15, administering anti-PD-1 antibody did not negatively impact delivery of the oncolytic virus. Addition of a checkpoint inhibitor—anti-PD-1—modifies the ad-DCT prime, both in terms of tumor-specific CD8+ T cell frequency and quality in B16 tumor-bearing animals. Addition of anti-PD-1 also enhances the Maraba-DCT boost, as exemplified by tumor-specific CD8+ T cell counts (approximately twice as many Ag-specific T cells). Importantly, these beneficial effects of combination therapy were associated with a profound increase in survival when compared to prime/boost or anti-PD-1 treatment alone. No toxic side effects were observed for the combination therapy nor did combination therapy negatively affect delivery of the oncolytic virus.

### Example 3

### The Combination of MG1 and Immune Checkpoint Inhibitor is Greatly Improves Efficacy in a Triple Negative Breast Cancer Model

Background

Triple-negative breast cancer is an aggressive systemic disease for which limited treatments are available. Triple-negative breast cancers (TNBC) are negative for the expression of the estrogen receptor, progesterone receptor and human epidermal growth factor receptor-2 and thus are refractory to conventional endocrine treatments including Tamoxifen and Trastuzumab which are commonly used for hormone-sensitive breast cancers (Hudis, C. A. & Gianni, L. Triple-negative breast cancer: an unmet medical need. Oncologist 16 Suppl 1, 1-11 (2011)) and the disseminated nature of late-stage forms further complicates treatment. The lack of options for patients with chemotherapy-resistant forms is pushing forward the rapid development of alternative strategies.

Using the clinical trial candidate rhabdovirus Maraba MG1, the importance of this immune response for TNBC treatment is demonstrated. Development of a clinically relevant model is described in which animals are re-challenged with orthotopic tumors following surgical resection of treated primary tumors. To mimic the recurrence of the disease in a clinically relevant setting for TNBC, development of a murine model of forced relapse is described in which primary tumors are treated with MG1 prior to surgical resection and implantation of new tumors. The virus induces an efficient tumor-specific immune response and recruits immune cells to the tumor. Importantly, the treatment with MG1 causes the induction of PDL1 by tumor cells and active regulatory T cells were found in greater amounts in the tumors.

Methods

Cell Lines and Culture

Vero kidney epithelial, 4T1 and EMT6 murine mammary carcinoma cell lines were purchased from the American Type Culture Collection (Manassas, Va.). Cells were maintained in Dulbecco's Modified Eagle's Medium (DMEM) (Corning cellgro, Manassas, Va.) supplemented with 10% fetal bovine serum (FBS) (Sigma life science, St-Louis, Mo.) and cultured at 37° C. with 5% CO₂.

Virus Production and Quantification

The expansion and purification of MG1-GFP was previously described (Brun, J. et al. Identification of genetically modified Maraba virus as an oncolytic rhabdovirus. Mol. Ther. 18, 1440-9 (2010)). Briefly, Vero cells were infected at an MOI of 0.01 for 24h prior to harvesting, filtration (0.22 μm bottle top filter (Millipore, Mass., USA)) and centrifugation (90 minutes at 30100 g) of the culture supernatant. The pellet was resuspended in Dulbecco's phosphate buffered saline (DPBS) (Corning cellgro, Manassas, Va.) and stored at −80° C. Viral titers were determined by plaque assay. Briefly, serially diluted samples were transferred to monolayers of Vero cells, incubated for 1 h and then overlaid with 0.5% agarose/DMEM supplemented with 10% FBS. Plaques were counted 24h later. In some experiments the virus was irradiated by exposure to 120mJ/cm² for 2 minutes using a Spectrolinker XL-1000 UV crosslinker as described previously (Zhang, J. et al. Maraba MG1 virus enhances natural killer cell function via conventional dendritic cells to reduce postoperative metastatic disease. Mol. Ther. 22, 1320-32 (2014)).

Microarray Analysis

Monolayers of 4T1 or EMT6 cells were treated at an MOI of 3 for 24h with either MG1-GFP or irradiated MG1-GFP. Culture supernatants were collected for CBA and ELISA analysis and the RNA was extracted from the cells using the RNeasy RNA extraction kit (Qiagen). Duplicate total RNA samples were processed and analysed on a MoGene2.0-st Affymetrix chip. Raw files were analyzed using the Transcriptome Analysis Console v3.0 (Affymetrix) software. Normalized transcript expression values further processed with R. Heatmaps were produced using the R package “pheatmap” v1.0.8. GO Term Enrichment analysis was performed using the online EnrichR tool (PMID 27141961). Genes selected for enrichment analysis are the subset of genes upregulated by MG1 infection relative to non-infected cells by at least 4-fold.

Flow Cytometry Analysis

Splenocytes were processed as previously described (Roy, D. G. et al. Programmable insect cell carriers for systemic delivery of integrated cancer biotherapy. J. Control. Release 220, 210-221 (2015)). Briefly, spleens were harvested and mashed through a 70 μm strainer (Fisher Scientific, Waltham, Mass.) prior to lysis of red blood cells using ACK lysis buffer and resuspension in FACS buffer (PBS, 3% FBS). For tumor cell extraction, we used the mouse tumor cocktail (Miltenyi) according the manufacturer's protocol with gentleMACS tubes and a gentleMACS Dissociator (Miltenyi). Cells were stained using various combinations of CD45, CD3, CD4, FoxP3 and PDL1 (all from BD Bioscience) and fixed using IC fixation buffer (eBioscience). For intranuclear staining, the FoxP3 staining buffer set was used (eBioscience). Flow cytometry analysis was performed on a Cyan ADP 9 (Beckman Coulter, Mississauga, ON).

In Vivo Experiments and Tumor Models

4T1 tumors were implanted into Balb/c mice (Charles River Laboratories). For the orthotopic models, 1×10⁵ cells were injected into the second right mammary fat pad. For treatments, the virus 1×10⁸ (plaque forming units—pfu) in a total volume of 100 uL of PBS was injected intratumorally (IT) or intravenously (IV) at the indicated time points using insulin syringes (The Stevens Co, Montreal, QC). The immune checkpoint inhibitors (anti-PD1 (clone RMPI-14, BioXcell) and anti-CTLA4 (clone 9D9, BioXcell)) were injected intraperitoneally (IP) at a dose of 100m each every second day for a total of 5 injections. For the tumor rechallenge model, 1×10⁵ cells were injected subcutaneously to the left flank of the animals. The tumors were treated at the indicated time points and resected 7 days after the first treatment. Four days after surgery, a higher dose of tumor cells (3×10⁵ cells) was seeded into the second right fat pad. The subset of mice that were rechallenged a second time more than 100 days post-tumor seeding were injected with 3×10⁵ EMT6 and 4T1 cells intra fat-pad bi-laterally.

Results

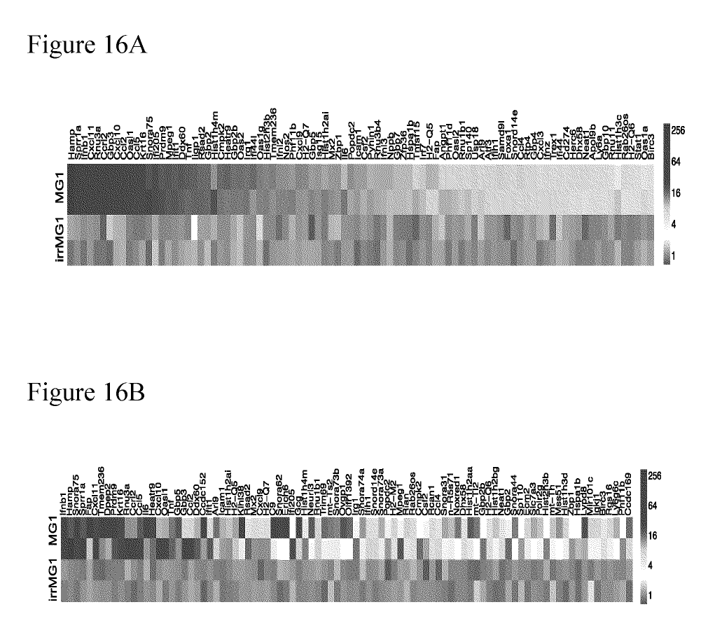

Pro-inflammatory signals are required to activate immune cells, but often also trigger the expression of the immune checkpoint inhibitor (ICI) PDL1 (Ritprajak, P. & Azuma, M. Intrinsic and extrinsic control of expression of the immunoregulatory molecule PD-L1 in epithelial cells and squamous cell carcinoma. Oral Oncol. 51, 221-228 (2015)). In order to shed light into the mechanisms by which the virus induces anti-tumor immunity, we performed a microarray analysis of 4T1 and EMT6 tumor cells infected in vitro with virus or irrMG1. Surprisingly, our results demonstrate that irrMG1 weakly induces only a few genes, which is in sharp contrast with MG1 which upregulates numerous genes at levels up to 300-fold higher then uninfected cells. Microarray analysis also showed the upregulation of PDL1 by both 4T1 and EMT6 cells with MG1 treatment respectively (FIG. 16A, and FIG. 16B).

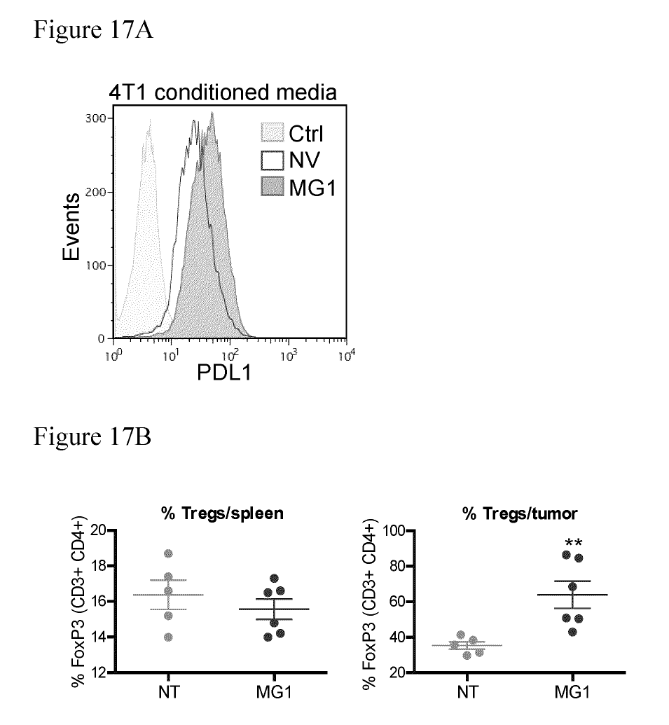

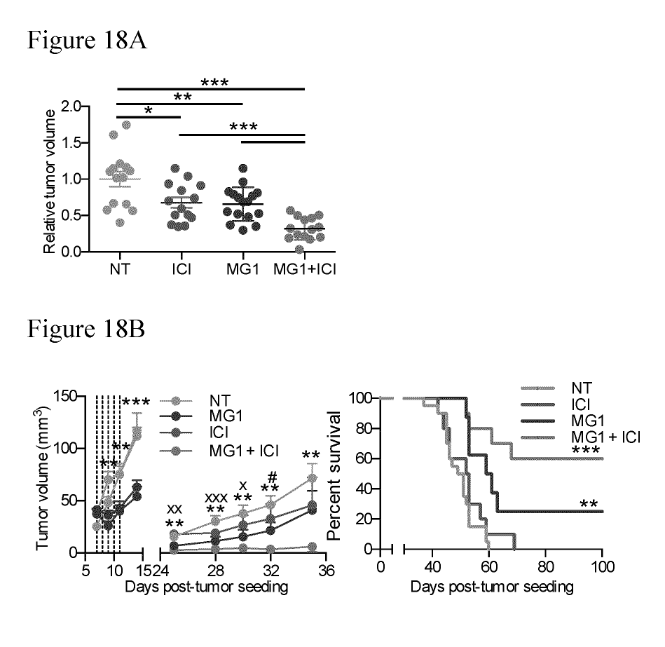

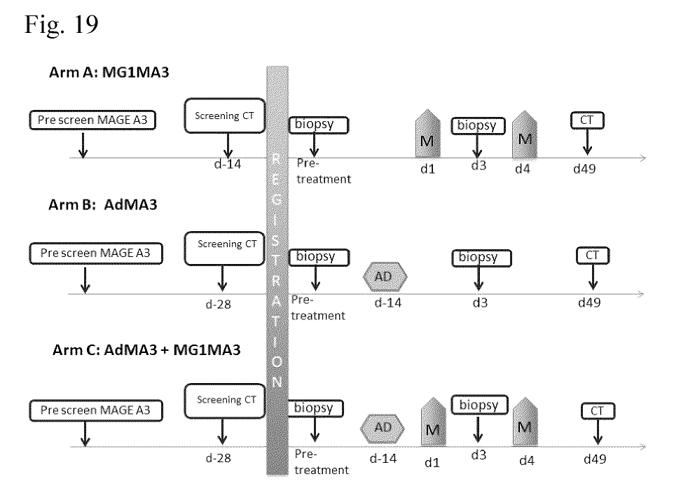

Additionally, virus-cleared 4T1 conditioned media induced the surface expression of PDL1 as determined by flow cytometry (FIG. 17A). We then assessed the presence of Tregs (CD3+, CD4+, FoxP3+cells) in treated animals and observed that, 10 days post-virus treatment, the percentage of Tregs remained unchanged in the spleen of the animals while the numbers increased from a little less then 40% to more then 60% of T cells in the tumors (FIG. 17B). Given the recent success of the ICIs in the clinic, as well as the various reports suggesting that pre-existing anti-tumor immunity is required for ICI treatment to be efficient and our data indicating that MG1 treatment induces a tumor-specific immune response, we sought to determine if the combination of both therapies could further improve outcomes. We tried to combine MG1 with both anti-PD1 and anti-CTLA4 treatments. In the orthotopic 4T1 model, we observed a significant reduction in the volume of tumors collected 12 days post-virus treatment, with the smallest being the tumors from the animals that received both MG1 and ICI treatments (FIG. 18A). Although the results appeared promising, no cures or survival advantages were observed using this treatment regimen (not shown). When using the tumor rechallenge model where the first tumors are treated or not with MG1 and the second tumors are only treated with the ICIs, we observed an important improvement in the tumor control as well as 60% cures for the group that received both treatments (FIG. 18B). This suggests that treating breast cancer patients with MG1 prior to surgery generates a protective immune response that can be further enhanced by ICI therapy in the case of a relapse.

Interestingly, the increased PDL1 expression as well as the accumulation of Tregs following MG1 treatment (FIGS. 17A, 17B and 18A), provides the opportunity for combination with ICI therapy. By reaching 60% cures in the 4T1 tumor model (FIG. 18B), we believe that the MG1-ICI combination is extremely promising. It is noteworthy that the ICI therapy on its own, while reducing the primary tumor burden, does not confer any survival advantage but greatly potentiates the pre-existing MG1-induced efficacy. This finding is in line with the various reports suggesting that pre-existing anti-tumor immune responses are required for efficient ICI treatment.

While cytokines and chemokines are induced by virus treatment, the immune checkpoint inhibitor (ICI) molecule PDL1 is also upregulated by tumor cells following MG1 infection. Given that virus treatment induces an anti-tumor immune response, cancers that would otherwise be refractory to ICI therapy could now be rendered sensitive. Given the recent success of ICI therapy, we investigated if the combination with this second treatment could further improve outcomes. Data demonstrates that the combination of MG1 with ICIs effectively cured most of the animals. The combination of both treatments increased survival to 60% in the aggressive 4T1 TNBC murine model.

### Example 4

### PDL Expression Levels in Tumor Biopsies Form Patients Pre- and Post-Treatment with an Oncolytic Virus Vaccine

Background

MG1MA3 is an RNA oncolytic virus (Maraba Rhabdovirus MG1) expressing human MAGE-A3 (transgenic MAGE-A3 insertion) that has the potential to selectively kill cancer cells through at least two major mechanisms. These include selective viral replication in cancer cells through a defective interferon response relative to normal cells. In addition to the replication of this virus in cancer cells the virus has also been engineered to express MAGE-A3 tumor associated antigens. Thus the host will generate a T cell immune response to this tumor antigen at the same time that the host immune system responds to the foreign viral protein. This immune response is considerably amplified if another virus (AdMA3; replication-defective, E1- and E3-deleted adenovirus serotype 5 with a transgene encoding human MAGE-A3) is used to initiate or “prime” a specific immune response to the MAGE-A3 tumor antigen prior to delivery of MG1MA3. The oncolytic virus vaccine leads to increased efficacy of MG1MA3.

Oncolytic Virus Vaccine Clinical Trial

Inclusion Criteria

A Phase I/II study of MG1 Maraba/MAGE-A3 (MG1MA3) with and Without Adenovirus Vaccine (AdMA3) was initiated in patients with incurable advanced/metastatic MAGE-A3-expressing solid tumors. In phase 1, enrolled patients have histologically confirmed, unresectable locally advanced/metastatic solid tumors with positive expression of MAGE-A3 (primary or metastatic lesion) and for which there is no known life prolonging standard therapy. In phase II, enrolled patients have histologically confirmed, unresectable locally advanced/metastatic solid tumors with positive expression of MAGE-A3 (primary or metastatic lesion) as follows: Non-small cell lung cancer (NSCLC) specifically adenocarcinoma and squamous cell carcinoma; Breast cancer that is ER/PR− HER2+; triple negative; ER and/or PR+HER2; Esophageal/GEJ (gastro-esophageal junction) cancer

Trial Design;

Arm A—MG1MA3 (virus) alone—patients receive a starting dose of MG1MA3 at a dose level of 1×10¹⁰ pfu administered IV on day 1 and day 4. MG1MA3 dose is escalated until a Dose Limiting Toxicity (DLT) is reached. Arm B—AdMA3 (vaccine prime) alone—patients receive prime AdMA3 vaccine at a dose of 1×10¹⁰ pfu administered IM on day (−14). No dose escalation is planned. Arm C—AdMA3 plus MG1MA3 (prime+boost)—patients receive prime AdMA3 vaccine administered as a single dose of 1×10¹⁰ pfu IM on day (−14) followed by dose escalation of MG1MA3 boost, IV administered on day 1 and day 4 at a starting dose of 1 log below the recommended Maximum Tolerated Dose (MTD) as determined in Arm A of the study. MG1MA3 dose will be escalated until a DLT is reached in a majority of the patients receiving that dose. For arms A and C a minimum of 3 patients are entered at each dose level, until the MTD is reached. Core/excisional tumor biopsies will be taken pre-treatment and post-treatment and analyzed for changes in gene expression of key markers in the tumor microenvironment including PDL1.

Methods

RNA was extracted from core patient biopsies using RNEasy Fibrous Tissue Mini Kit as per kit protocol (Qiagen, 74704). Briefly, tissue was disrupted in RLT buffer using Qiagen TissueRuptor homogenizer. RNA was then extracted using an automated QIAcube sample preparation as per protocol. Following extraction RNA was quantified on a 2100 Bioanalyzer (Agilent Technologies) and then up to 100 μg was used for analysis using a custom Nanostring Elements CodeSet and nCounter 144-plex Elements TagSet. The resulting data was analyzed using nCounter analysis software (Nanostring Technologies).

Results

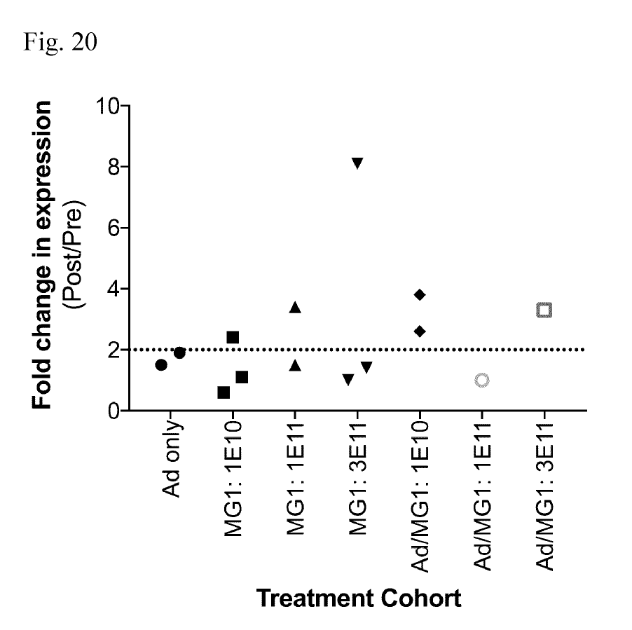

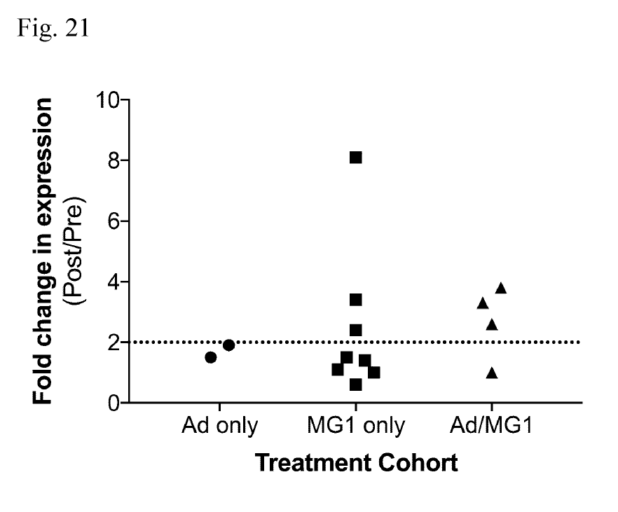

Clinical PDL1 expression data was generated by NanoString analysis of tumor biopsies pre-treatment and two days post-treatment after the first dose of MG1. NanoString analysis looks at PDL1 transcript levels, results were expressed as fold change in pre-treatment levels versus post-treatment levels and calculated by dividing post-treatment expression levels by pre-treatment expression levels and graphed 2 different ways. FIG. 20 shows the fold change in PDL1 levels in individual tumor biopsies (Post-treatment versus Pre-treatment) at each dose in Arms A (Ad only), B (MG1 only) and C (Ad/MG1) of the current clinical trial. FIG. 21 shows the fold change in PDL1 levels from pooled tumor biopsies (Post-treatment versus Pre-treatment) for all doses in Arms A (Ad only), B (MG1 only) and C (Ad/MG1) in current clinical trial. The data demonstrates that MG1 and Ad/MG1 treatment leads to an increase in PDL1 expression in the tumors in a number of patients, supporting a combination therapy with a checkpoint inhibitor according to the methods herein described.

### Example 5

### Oncolytic Virus Vaccine Plus Checkpoint Inhibitor Combination Treatment Clinical Trial

A phase I/II, multicenter, open-label clinical trial of MG1 Maraba/MAGE-A3 (MG1MA3) with adenovirus vaccine with trangenic MAGE-A3 insertion (AdMA3) (prime:boost regimne) in combination with Pembrolizumab in patients with previously treated metastatic non-small cell lung cancer (NSCLC) is described. MG1MA3 and Pembrolizumab will be administered as standard therapies.

Patients will have histological subtype squamous or non-squamous NSCLC tumors with positive expression of MAGE-A3 (primary or metastatic lesion) who have completed a first standard therapy with a platinum-based chemotherapy.

Patients will receive a single dose of prime AdMA3 vaccine at a dose of 1×10¹⁰ pfu administered intramuscularly (IM) on day (−14) and will be administered MG1MA3 by IV infusion at a dose level of 1×10¹⁰ pfu on day 1 and day 4 (boost). If this dose is tolerated in combination with pembrolizumab, a second cohort will be treated with 1×10¹¹ MG1MA3 on day 1 and 4. Patients will receive Prembrolizumab at a dose of 200 mg IV on day (−13), day 8, and every 3 weeks thereafter until confirmed radiographic progression is observed. Tumor biopsies will be taken pre-treatment and post-treatment and analyzed for changes in gene expression of key markers in the tumor microenvironment including PDL1. The objective tumor response rate (ORR) based on RECIST v1.1 will be evaluated in phase 2.

APPENDIX A
Protein and Nucleotide Sequences

Protein sequence of full length, wild type, human
MAGEA3 (SEQ ID NO: 35):
MPLEQRSQHCKPEEGLEARGEALGLVGAQAPATEEQEAASSSSTLVEVTL
GEVPAAESPDPPQSPQGASSLPTTMNYPLWSQSYEDSSNQEEEGPSTFPD
LESEFQAALSRKVAELVHFLLLKYRAREPVTKAEMLGSWGNWQYFFPVIF
SKASSSLQLVFGIELMEVDPIGHLYIFATCLGLSYDGLLGDNQIMPKAGL
LIIVLAIIAREGDCAPEEKIWEELSVLEVFEGREDSILGDPKKLLTQHYV
QENYLEYRQVPGSDPACYEFLWGPRALVETSYVKVLHHMVKISGGPHISY
PPLHEWVLREGEE*
DNA sequence encoding full length, wild type,
human MAGEA3 (SEQ ID NO: 36):
ATGCCTCTTGAGCAGAGGAGTCAGCACTGCAAGCCTGAAGAAGGCCTTGA
GGCCCGAGGAGAGGCCCTGGGCCTGGTGGGTGCGCAGGCTCCTGCTACTG
AGGAGCAGGAGGCTGCCTCCTCCTCTTCTACTCTAGTTGAAGTCACCCTG
GGGGAGGTGCCTGCTGCCGAGTCACCAGATCCTCCCCAGAGTCCTCAGGG
AGCCTCCAGCCTCCCCACTACCATGAACTACCCTCTCTGGAGCCAATCCT
ATGAGGACTCCAGCAACCAAGAAGAGGAGGGGCCAAGCACCTTCCCTGAC
CTGGAGTCCGAGTTCCAAGCAGCACTCAGTAGGAAGGTGGCCGAGTTGGT
TCATTTTCTGCTCCTCAAGTATCGAGCCAGGGAGCCGGTCACAAAGGCAG
AAATGCTGGGGAGTGTCGTCGGAAATTGGCAGTATTTCTTTCCTGTGATC
TTCAGCAAAGCTTCCAGTTCCTTGCAGCTGGTCTTTGGCATCGAGCTGAT
GGAAGTGGACCCCATCGGCCACTTGTACATCTTTGCCACCTGCCTGGGCC
TCTCCTACGATGGCCTGCTGGGTGACAATCAGATCATGCCCAAGGCAGGC
CTCCTGATAATCGTCCTGGCCATAATCGCAAGAGAGGGCGACTGTGCCCC
TGAGGAGAAAATCTGGGAGGAGCTGAGTGTGTTAGAGGTGTTTGAGGGGA
GGGAAGACAGTATCTTGGGGGATCCCAAGAAGCTGCTCACCCAACATTTC
GTGCAGGAAAACTACCTGGAGTACCGGCAGGTCCCCGGCAGTGATCCTGC
ATGTTATGAATTCCTGTGGGGTCCAAGGGCCCTCGTTGAAACCAGCTATG
TGAAAGTCCTGCACCATATGGTAAAGATCAGTGGAGGACCTCACATTTCC
TACCCACCCCTGCATGAGTGGGTTTTGAGAGAG GGGGAAGAGTGA
Codon optimized DNA sequence encoding full length,
wild type, human MAGEA3 protein (SEQ ID NO: 37):
ATGCCCCTGGAGCAGCGGTCTCAGCATTGCAAGCCAGAGGAGGGCCTCGA
GGCGAGGGGCGAGGCCCTCGGCTTGGTGGGGGCGCAGGCTCCTGCAACCG
AGGAGCAAGAGGCCGCATCCAGTTCCTCTACCCTGGTTGAGGTGACCTTG
GGTGAGGTGCCCGCCGCGGAGAGCCCCGACCCGCCTCAAAGCCCCCAGGG
TGCCAGCTCCCTGCCCACAACAATGAACTACCCACTCTGGAGTCAGTCTT
ACGAGGACAGTAGTAACCAAGAGGAGGAGGGACCCTCCACATTCCCAGAC
CTGGAGTCTGAATTCCAGGCAGCATTGTCTAGAAAAGTGGCCGAATTGGT
GCACTTCCTGCTGCTGAAGTATCGCGCCCGCGAGCCAGTCACAAAAGCTG
AAATGCTGGGTTCTGTCGTGGGAAATTGGCAGTACTTCTTCCCCGTGATC
TTCAGTAAAGCGTCCAGCTCCTTGCAGCTGGTCTTTGGTATCGAGCTGAT
GGAGGTGGATCCCATCGGCCATCTGTATATCTTTGCCACATGCCTGGGCC
TGAGCTACGATGGCCTGCTGGGCGACAACCAGATCATGCCAAAAGCTGGC
CTGCTGATCATCGTTCTGGCTATCATCGCTAGAGAAGGAGATTGCGCCCC
TGAAGAAAAGATCTGGGAGGAACTGAGCGTCCTGGAAGTCTTTGAGGGTC
GTGAAGACAGCATTCTCGGGGATCCCAAGAAGCTGCTGACCCAGCACTTC
GTGCAGGAGAACTATCTGGAGTACCGCCAGGTTCCCGGCAGCGACCCCGC
TTGCTACGAGTTCCTGTGGGGCCCCAGGGCCCTGGTCGAGACATCCTACG
TGAAGGTCCTGCACCATATGGTTAAAATCAGCGGCGGCCCCCATATCTCT
TATCCGCCGCTCCACGAGTGGGTGCTCC GGGAGGGAGAGGAG
Protein sequence of a variant of full length, wild
type, human MAGEA3 (SEQ ID NO: 38):
MPLEQRSQHCKPEEGLEARGEALGLVGAQAPATEEQEAASSSSTLVEVTL
GEVPAAESPDPPQSPQGASSLPTTMNYPLWSQSYEDSSNQEEEGPSTFPD
LESEFQAALSRKVAELVHFLLLKYRAREPVTKAEMLGSWGNWQYFFPVIF
SKASSSLQLVFGIELMEVDPIGHLYIFATCLGLSYDGLLGDNQIMPKAGL
LIIVLAIIAREGDCAPEEKIWEELSVLEVFEGREDSILGDPKKLLTQFIF
VQENYLEYRQVPGSDPACYEFLWGPRALVETSYVKVLBHMVKISGGPHIS
YPPLBEWVL REGEEDYKDDDDK*
DNA sequence encoding a variant of full length,
wild type, human MAGEA3 (SEQ ID NO: 39):
ATGCCCCTGGAACAGCGGAGCCAGCACTGCAAGCCCGAGGAAGGCCTGGA
AGCCAGAGGCGAAGCCCTGGGACTGGTGGGAGCCCAGGCCCCTGCCACAG
AAGAACAGGAAGCCGCCAGCAGCAGCTCCACCCTGGTGGAAGTGACCCTG
GGCGAAGTGCCTGCCGCCGAGAGCCCTGATCCCCCTCAGTCTCCTCAGGG
CGCCAGCAGCCTGCCCACCACCATGAACTACCCCCTGTGGTCCCAGAGCT
ACGAGGACAGCAGCAACCAGGAAGAGGAAGGCCCCAGCACCTTCCCCGAC
CTGGAAAGCGAGTTCCAGGCCGCCCTGAGCCGGAAGGTGGCAGAGCTGGT
GCACTTCCTGCTGCTGAAGTACAGAGCCCGCGAGCCCGTGACCAAGGCCG
AGATGCTGGGCAGCGTGGTGGGAAACTGGCAGTACTTCTTCCCCGTGATC
TTCTCCAAGGCCAGCAGCTCCCTGCAGCTGGTGTTCGGCATCGAGCTGAT
GGAAGTGGACCCCATCGGCCACCTGTACATCTTCGCCACCTGTCTGGGCC
TGAGCTACGACGGCCTGCTGGGCGACAACCAGATCATGCCCAAGGCCGGC
CTGCTGATCATCGTGCTGGCCATCATTGCCCGCGAGGGCGACTGCGCCCC
TGAGGAAAAGATCTGGGAGGAACTGAGCGTGCTGGAAGTGTTCGAGGGCA
GAGAGGACAGCATCCTGGGCGACCCCAAGAAGCTGCTGACCCAGCACTTC
GTGCAGGAAAACTACCTGGAATACCGCCAGGTGCCCGGCAGCGACCCCGC
CTGTTACGAGTTCCTGTGGGGCCCCAGGGCTCTGGTGGAAACCAGCTACG
TGAAGGTGCTGCACCACATGGTGAAAATCAGCGGCGGACCCCACATCAGC
TACCCCCCACTGCACGAGTGGGTGCTGAGAGAGGGCGAAGAGGACTACAA
GGACGACGACGACAAATGA
Protein sequence of HPV E6/E7 fusion protein (SEQ
ID NO: 40):
MHQKRTAMFQDPQERPRKLPQLCTELQTTIHDIILECVYCKQQLLRREVY
DFAFRDLCIVYRDGNPYAVDKLKFYSKISEYRHYCYSVYGTTLEQQYNKP
LCDLLIRINQKPLCPEEKQRFILDKKQRFFINIRGRWTGRCMSCCRSSRT
RRETQLGGGGGAAYMARFEDPTRRPYKLPDLCTELNTSLQDIEITCVYCK
TVLELTEVFEFAFKDLFWYRDSIPHAAFIKIDFYSRIRELRHYSDSVYGD
TLEKLTNTGLYNLLIRLRQKPLNPAEKLRFILNEKRRFFINIAGHYRGQC
HSCCNRARQERLQRRRETQVGGGGGAAYMEGDTPTLHEYMLDLQPETTDL
YQLNDSSEEEDEIDGPAGQAEPDRAHYNIVTFCCKCDSTLRLCVQSTHVD
IRTLEDLLMGTLGIVPICSQKPGGGGGAAYMITGPKATLQDIVLHLEPQN
EIPVDLLQLSDSEEENDEIDGVNHQHLPARRAEPQRHTMLCMCCKCEARI
KLWESSADDLRAFQQLFLNTLSFVPWCASQQ*
DNA sequence of HPV E6/E7 fusion protein (SEQ ID
NO: 41):
ATGCATCAGAAGCGAACTGCTATGTTTCAGGACCCTCAGGAGCGGCCACG
CAAACTGCCTCAGCTGTGCACCGAACTGCAGACAACTATCCACGACATCA
TTCTGGAATGCGTGTACTGTAAGCAGCAGCTGCTGAGGAGAGAGGTCTAT
GACTTCGCTTTTCGCGATCTGTGCATCGTGTACCGAGACGGAAACCCATA
TGCAGTCGATAAGCTGAAGTTCTACAGCAAGATCTCCGAATACAGGCATT
ACTGTTACAGCGTGTACGGGACCACACTGGAGCAGCAGTATAACAAGCCC
CTGTGCGACCTGCTGATCAGAATTAATCAGAAGCCCCTGTGCCCTGAGGA
AAAACAGAGGCACCTGGATAAGAAACAGAGATTTCATAACATCCGAGGAC
GATGGACCGGGCGGTGCATGTCCTGCTGTAGAAGCTCCCGGACTCGACGA
GAGACCCAGCTGGGCGGAGGAGGAGGAGCAGCTTACATGGCACGATTCGA
GGACCCTACCCGAAGGCCATATAAGCTGCCCGACCTGTGCACAGAACTGA
ATACTTCTCTGCAGGACATCGAGATTACATGCGTGTACTGTAAAACCGTC
CTGGAGCTGACAGAAGTGTTCGAGTTTGCTTTCAAGGACCTGTTTGTGGT
CTACCGGGATTCAATCCCTCACGCAGCCCATAAAATCGACTTCTACAGCA
GGATCAGGGAACTGCGCCACTACTCCGACAGCGTGTACGGGGATACACTG
GAGAAGCTGACAAACACTGGCCTGTACAATCTGCTGATCCGACTGCGACA
GAAGCCACTGAACCCAGCCGAAAAACTGAGACACCTGAACGAGAAGAGAC
GGTTTCACAATATTGCAGGCCATTATAGGGGACAGTGCCATAGTTGCTGT
AATCGAGCCAGGCAGGAAAGACTGCAGCGCCGAAGGGAGACTCAAGTCGG
CGGAGGAGGAGGAGCTGCATACATGCACGGCGACACCCCCACACTGCATG
AATATATGCTGGATCTGCAGCCTGAGACTACCGACCTGTACCAGCTGAAC
GATTCTAGTGAGGAAGAGGACGAAATCGACGGACCAGCAGGACAGGCAGA
GCCTGACCGGGCCCACTATAATATTGTGACATTCTGCTGTAAGTGCGATT
CTACTCTGCGGCTGTGCGTGCAGAGTACTCATGTCGACATCCGCACCCTG
GAGGATCTGCTGATGGGGACTCTGGGCATCGTCCCAATTTGTAGCCAGAA
ACCAGGCGGCGGCGGCGGAGCAGCTTACATGCACGGACCCAAGGCTACCC
TGCAGGACATCGTGCTGCATCTGGAACCTCAGAATGAGATTCCAGTCGAC
CTGCTGCAGCTGAGTGATTCAGAAGAGGAAAACGACGAGATCGACGGCGT
GAATCACCAGCATCTGCCTGCTAGACGGGCAGAGCCACAGCGACACACAA
TGCTGTGCATGTGCTGTAAGTGTGAAGCCAGGATCAAGCTGGTGGTCGAG
TCAAGCGCCGACGATCTGCGCGCCTTCCAGCAGCTGTTCCTGAATACTCT
GTCATTTGTCCCTTGGTGTGCCTC CCAGCAGTGA
Protein sequence of huSTEAP protein (SEQ ID
NO: 42):
MESRKDITNQEELWKMKPRRNLEEDDYLHKDTGETSMLKRPVLLHLHQTA
HADEFDCPSELQHTQELFPQWHLPIKIAAIIASLTFLYTLLREVIHPLAT
SHQQYFYKIPILVINKVLPMVSITLLALVYLPGVIAAIVQLFINGTKYKK
FPHWLDKWMLTRKQFGLLSFFFAVLHAIYSLSYPMRRSYRYKLLNWAYQQ
VQQNKEDAWIEHDVWRMEIYVSLGIVGLAILALLAVTSIPSVSDSLTWRE
FHYIQSKLGIVSLLLGTIHALIFAWNKWIDIKQFVWYTPPTFMIAVFLPI
WLIFKSILFLPCLRKKILKIRHGWEDVTKINKTEICSQLKL*
DNA sequence of huSTEAP protein (SEQ ID NO: 43):
ATGGAATCACGGAAGGACATCACTAATCAGGAGGAACTGTGGAAAATGAA
GCCAAGAAGGAATCTGGAAGAGGACGACTATCTGCACAAGGACACCGGCG
AAACAAGTATGCTGAAACGACCAGTGCTGCTGCACCTGCATCAGACTGCT
CACGCAGACGAGTTTGATTGCCCCTCTGAACTGCAGCACACCCAGGAGCT
GTTCCCACAGTGGCATCTGCCCATCAAGATTGCCGCTATCATTGCTTCAC
TGACATTTCTGTACACTCTGCTGAGAGAAGTGATCCACCCCCTGGCCACC
AGCCATCAGCAGTACTTCTATAAGATCCCTATCCTGGTCATCAACAAGGT
CCTGCCAATGGTGAGCATCACACTGCTGGCCCTGGTCTACCTGCCTGGAG
TGATCGCAGCCATTGTCCAGCTGCACAATGGGACAAAGTATAAGAAATTT
CCACATTGGCTGGATAAGTGGATGCTGACTAGGAAACAGTTCGGACTGCT
GTCCTTCTTTTTCGCCGTGCTGCACGCTATCTACAGCCTGTCCTATCCCA
TGAGGAGGAGCTACCGGTATAAGCTGCTGAACTGGGCTTACCAGCAGGTG
CAGCAGAACAAGGAGGACGCATGGATTGAACATGACGTGTGGCGCATGGA
AATCTACGTGAGCCTGGGCATTGTCGGACTGGCCATCCTGGCTCTGCTGG
CAGTGACCAGTATCCCTTCTGTCAGTGACTCACTGACATGGAGAGAGTTT
CACTACATTCAGAGCAAGCTGGGGATCGTGTCCCTGCTGCTGGGCACCAT
CCATGCACTGATTTTTGCCTGGAACAAGTGGATCGATATCAAGCAGTTCG
TGTGGTATACTCCCCCTACCTTTATGATTGCCGTCTTCCTGCCCATCGTG
GTCCTGATCTTCAAGTCCATCCTGTTCCTGCCTTGTCTGCGGAAGAAAAT
CCTGAAAATTCGGCACGGATGGGAGGATGTCACCAAAATCAATAAGACTG
AAATCTGTAGCCAGCTGAAGCTTTAA
Protein sequence of NYESQ1 MAR protein (SEQ ID
NO: 44):
MQAEGRGTGGSTGDADGPGGPGIPDGPGGNAGGPGEAGATGGRGPRGAGA
ARASGPGGGAPRGPHGGAASGLNGCCRCGARGPESRLLEFYLAMPFATPM
EAELARRSLAQDAPPLPVPGVLLKEFTVSGNILTIRLTAADHRQLQLSIS
SCLQQLSLLMWITQCFLPVFLAQPPSGQRR*
DNA sequence of NYES01 MAR (SEQ ID NO: 45):
ATGCAGGCCGAGGGCAGAGGCACAGGCGGATCTACAGGCGACGCCGATGG
CCCTGGCGGCCCTGGAATTCCTGACGGACCTGGCGGCAATGCCGGCGGAC
CCGGAGAAGCTGGCGCCACAGGCGGAAGAGGACCTAGAGGCGCTGGCGCC
GCTAGAGCTTCTGGACCAGGCGGAGGCGCCCCTAGAGGACCTCATGGCGG
AGCCGCCTCCGGCCTGAACGGCTGTTGCAGATGTGGAGCCAGAGGCCCCG
AGAGCCGGCTGCTGGAATTCTACCTGGCCATGCCCTTCGCCACCCCCATG
GAAGCCGAGCTGGCCAGACGGTCCCTGGCCCAGGATGCTCCTCCTCTGCC
TGTGCCCGGCGTGCTGCTGAAAGAATTCACCGTGTCCGGCAACATCCTGA
CCATCCGGCTGACTGCCGCCGACCACAGACAGCTCCAGCTGTCTATCAGC
TCCTGCCTGCAGCAGCTGAGCCTGCTGATGTGGATCACCCAGTGCTTTCT
GCCCGTGTTCCTGGCTCAGCCCCCCAGCGGCCAGAGAAGATGA

1. A method for treating and/or preventing cancer or prolonging an anti-tumor response in a mammal in need thereof, comprising administering to the mammal an effective amount of a combination comprising (a) a replicative oncolytic rhabdovirus and (b) one or more checkpoint inhibitors.

2. (canceled)

3. The method of claim 1, wherein the checkpoint inhibitor inhibits a checkpoint protein selected from the group consisting of: cytotoxic T-lymphocyte antigen-4 (CTLA4), programmed cell death protein 1 (PD-1), PD-L1, PD-L2, B7-H3, B7-H4, herpesvirus entry mediator (HVEM), T cell membrane protein 3 (TIM3), galectin 9 (GAL9), lymphocyte activation gene 3 (LAG3), V-domain immunoglobulin (Ig)-containing suppressor of T-cell activation (VISTA), Killer-Cell Immunoglobulin-Like Receptor (KIR), Band T lymphocyte attenuator (BTLA), T cell immunoreceptor with Ig and ITIM domains (TIGIT), and combinations thereof.

4. The method of claim 3, wherein the checkpoint inhibitor inhibits CTLA-4, PD-1 or PD-L1.

5. The method of claim 4, wherein the checkpoint inhibitor inhibits CTLA-4 and is selected from Ipilimumab and Tremelimumab.

6. The method of claim 4, wherein the checkpoint inhibitor inhibits PD-1 and is selected from Nivolumab, Pembrolizumab, Pidilizumab, lambrolizumab, and AMP-224.

7. The method of claim 4, wherein the checkpoint inhibitor inhibits PD-L1 and is selected from BMS-936559, MEDI-4736, MPDL33280A, MIHI, Atezolizumab, Durvalumab and Avelumab.

8. The method of claim 1, wherein the oncolytic rhabdovirus is administered to the mammal in combination with at least two checkpoint inhibitors.

9. The method of claim 1, wherein the oncolytic rhabdovirus and the checkpoint inhibitor are administered simultaneously.

10. The method of claim 1, wherein the oncolytic rhabdovirus and the checkpoint inhibitor are administered sequentially and wherein a first administration of checkpoint inhibitor occurs prior to a first administration of oncolytic virus and preferably occurs within 30 days of a first administration of oncolytic virus.

11. The method of claim 1, wherein the oncolytic rhabdovirus expresses a tumor associated antigen.

12. The method of claim 11, wherein the tumor associated antigen is selected from the group consisting of MAGEA3, Human Papilloma Virus E6/E7 fusion protein, human Six-Transmembrane Epithelial Antigen of the Prostate protein, Cancer Testis Antigen 1, and a variant thereof.

13. The method of claim 11, wherein the mammal has a pre-existing immunity to the tumor associated antigen.

14. The method of claim 13, wherein the pre-existing immunity in the mammal is established by administering said tumor associated antigen to the mammal prior to administering the oncolytic rhabodvirus.

15. The method of claim 14, wherein the pre-existing immunity in the mammal is established by administering an expression vector encoding said tumor associated antigen to the mammal prior to administering the oncolytic rhabdovirus.

16. (canceled)

17. The method of claim 1, wherein the oncolytic rhabdovirus is an oncolytic vesiculovirus.

18. The method of claim 17, wherein the oncolytic rhabdovirus is a wild type or genetically modified VSV or Maraba strain rhabdovirus.

19. The method of claim 17, wherein the oncolytic rhabdovirus is VSVdelta51 or Maraba MG1.

20. (canceled)

21. (canceled)

22. (canceled)

23. The method of claim 1, wherein the cancer is colorectal cancer, lung cancer, melanoma, pancreatic cancer, ovarian cancer, renal cell carcinoma, cervical cancer, liver cancer, breast cancer, head and neck cancer, prostate cancer, gastro-esophagael junction cancer, brain cancer, and soft tissue sarcoma.

24. The method of claim 23, wherein the cancer is ER/PR− HER2+breast cancer, triple negative breast cancer, ER and/or PR+HER2+breast cancer, squamous or non-squamous non-small cell lung cancer (NSCLC) or gastroesophageal junction cancer.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. The method of claim 1, wherein the oncolytic rhabdovirus and the checkpoint inhibitor are administered sequentially and wherein the first dose of oncolytic virus is administered prior to a first dose of the checkpoint inhibitor.


SEQUENCE LISTING (45 sequences)

SEQ ID NO: 1 - protein, 9 aa, Artificial Sequence
  antigenic epitope from human MAGEA3 protein
EVDPIGHLY

SEQ ID NO: 2 - protein, 9 aa, Artificial Sequence
  antigenic epitope from human MAGEA3 protein
FLWGPRALV

SEQ ID NO: 3 - protein, 9 aa, Artificial Sequence
  antigenic epitope from human MAGEA3 protein
KVAELVHFL

SEQ ID NO: 4 - protein, 9 aa, Artificial Sequence
  antigenic epitope from human MAGEA3 protein
TFPDLESEF

SEQ ID NO: 5 - protein, 9 aa, Artificial Sequence
  antigenic epitope from human MAGEA3 protein
VAELVHFLL

SEQ ID NO: 6 - protein, 10 aa, Artificial Sequence
  antigenic epitope from human MAGEA3 protein
MEVDPIGHLY

SEQ ID NO: 7 - protein, 9 aa, Artificial Sequence
  antigenic epitope from human MAGEA3 protein
EVDPIGHLY

SEQ ID NO: 8 - protein, 10 aa, Artificial Sequence
  antigenic epitope from human MAGEA3 protein
REPVTKAEML

SEQ ID NO: 9 - protein, 9 aa, Artificial Sequence
  antigenic epitope from human MAGEA3 protein
AELVHFLLL

SEQ ID NO: 10 - protein, 10 aa, Artificial Sequence
  antigenic epitope from human MAGEA3 protein
MEVDPIGHLY

SEQ ID NO: 11 - protein, 9 aa, Artificial Sequence
  antigenic epitope from human MAGEA3 protein
WQYFFPVIF

SEQ ID NO: 12 - protein, 9 aa, Artificial Sequence
  antigenic epitope from human MAGEA3 protein
EGDCAPEEK

SEQ ID NO: 13 - protein, 16 aa, Artificial Sequence
  antigenic epitope from human MAGEA3 protein
KKLLTQHFVQENYLEY

SEQ ID NO: 14 - protein, 15 aa, Artificial Sequence
  antigenic epitope from human MAGEA3 protein
RKVAELVHFLLLKYR

SEQ ID NO: 15 - protein, 16 aa, Artificial Sequence
  antigenic epitope from human MAGEA3 protein
KKLLTQHFVQENYLEY

SEQ ID NO: 16 - protein, 16 aa, Artificial Sequence
  antigenic epitope from human MAGEA3 protein
ACYEFLWGPRALVETS

SEQ ID NO: 17 - protein, 15 aa, Artificial Sequence
  antigenic epitope from human MAGEA3 protein
RKVAELVHFLLLKYR

SEQ ID NO: 18 - protein, 12 aa, Artificial Sequence
  antigenic epitope from human MAGEA3 protein
VIFSKASSSLQL

SEQ ID NO: 19 - protein, 12 aa, Artificial Sequence
  antigenic epitope from human MAGEA3 protein
VIFSKASSSLQL

SEQ ID NO: 20 - protein, 15 aa, Artificial Sequence
  antigenic epitope from human MAGEA3 protein
VFGIELMEVDPIGHL

SEQ ID NO: 21 - protein, 15 aa, Artificial Sequence
  antigenic epitope from human MAGEA3 protein
GDNQIMPKAGLLIIV

SEQ ID NO: 22 - protein, 15 aa, Artificial Sequence
  antigenic epitope from human MAGEA3 protein
TSYVKVLHHMVKISG

SEQ ID NO: 23 - protein, 16 aa, Artificial Sequence
  antigenic epitope from human MAGEA3 protein
RKVAELVHFLLLKYRA

SEQ ID NO: 24 - protein, 16 aa, Artificial Sequence
  antigenic epitope from human MAGEA3 protein
FLLLKYRAREPVTKAE

SEQ ID NO: 25 - protein, 9 aa, Artificial Sequence
  antigenic epitope from human MAGEA3 protein
FLWGPRALV

SEQ ID NO: 26 - protein, 9 aa, Artificial Sequence
  antigenic epitope from human MAGEA3 protein
KVAELVHFL

SEQ ID NO: 27 - protein, 9 aa, Artificial Sequence
  antigenic epitope from human MAGEA3 protein
EGDCAPEEK

SEQ ID NO: 28 - protein, 16 aa, Artificial Sequence
  antigenic epitope from human MAGEA3 protein
KKLLTQHFVQENYLEY

SEQ ID NO: 29 - protein, 15 aa, Artificial Sequence
  antigenic epitope from human MAGEA3 protein
RKVAELVHFLLLKYR

SEQ ID NO: 30 - protein, 16 aa, Artificial Sequence
  antigenic epitope from human MAGEA3 protein
KKLLTQHFVQENYLEY

SEQ ID NO: 31 - protein, 440 aa, Artificial Sequence
  Synthetic Antibody chain polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 214 aa, Artificial Sequence
  Synthetic Antibody chain polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 447 aa, Artificial Sequence
  Synthetic Antibody chain polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 218 aa, Artificial Sequence
  Synthetic Antibody chain polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 313 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSWGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 36 - DNA, 945 bp, Homo sapiens
atgcctcttgagcagaggagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgagttggttcattttctgctcctcaagtatcgagccagggagccggtcacaaaggcagaaatgctggggagtgtcgtcggaaattggcagtatttctttcctgtgatcttcagcaaagcttccagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagtga

SEQ ID NO: 37 - DNA, 942 bp, Artificial Sequence
  codon optimized nucleotide sequence encoding full length, wild type, human MAGEA3 protein
atgcccctggagcagcggtctcagcattgcaagccagaggagggcctcgaggcgaggggcgaggccctcggcttggtgggggcgcaggctcctgcaaccgaggagcaagaggccgcatccagttcctctaccctggttgaggtgaccttgggtgaggtgcccgccgcggagagccccgacccgcctcaaagcccccagggtgccagctccctgcccacaacaatgaactacccactctggagtcagtcttacgaggacagtagtaaccaagaggaggagggaccctccacattcccagacctggagtctgaattccaggcagcattgtctagaaaagtggccgaattggtgcacttcctgctgctgaagtatcgcgcccgcgagccagtcacaaaagctgaaatgctgggttctgtcgtgggaaattggcagtacttcttccccgtgatcttcagtaaagcgtccagctccttgcagctggtctttggtatcgagctgatggaggtggatcccatcggccatctgtatatctttgccacatgcctgggcctgagctacgatggcctgctgggcgacaaccagatcatgccaaaagctggcctgctgatcatcgttctggctatcatcgctagagaaggagattgcgcccctgaagaaaagatctgggaggaactgagcgtcctggaagtctttgagggtcgtgaagacagcattctcggggatcccaagaagctgctgacccagcacttcgtgcaggagaactatctggagtaccgccaggttcccggcagcgaccccgcttgctacgagttcctgtggggccccagggccctggtcgagacatcctacgtgaaggtcctgcaccatatggttaaaatcagcggcggcccccatatctcttatccgccgctccacgagtgggtgctccgggagggagaggag

SEQ ID NO: 38 - protein, 321 aa, Artificial Sequence
  variant of MAGEA3 protein derived from homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSWGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEDYKDDDDK

SEQ ID NO: 39 - DNA, 969 bp, Artificial Sequence
  nucleotide sequence encoding variant of MAGEA3 protein derived from homo sapiens
atgcccctggaacagcggagccagcactgcaagcccgaggaaggcctggaagccagaggcgaagccctgggactggtgggagcccaggcccctgccacagaagaacaggaagccgccagcagcagctccaccctggtggaagtgaccctgggcgaagtgcctgccgccgagagccctgatccccctcagtctcctcagggcgccagcagcctgcccaccaccatgaactaccccctgtggtcccagagctacgaggacagcagcaaccaggaagaggaaggccccagcaccttccccgacctggaaagcgagttccaggccgccctgagccggaaggtggcagagctggtgcacttcctgctgctgaagtacagagcccgcgagcccgtgaccaaggccgagatgctgggcagcgtggtgggaaactggcagtacttcttccccgtgatcttctccaaggccagcagctccctgcagctggtgttcggcatcgagctgatggaagtggaccccatcggccacctgtacatcttcgccacctgtctgggcctgagctacgacggcctgctgggcgacaaccagatcatgcccaaggccggcctgctgatcatcgtgctggccatcattgcccgcgagggcgactgcgcccctgaggaaaagatctgggaggaactgagcgtgctggaagtgttcgagggcagagaggacagcatcctgggcgaccccaagaagctgctgacccagcacttcgtgcaggaaaactacctggaataccgccaggtgcccggcagcgaccccgcctgttacgagttcctgtggggccccagggctctggtggaaaccagctacgtgaaggtgctgcaccacatggtgaaaatcagcggcggaccccacatcagctaccccccactgcacgagtgggtgctgagagagggcgaagaggactacaaggacgacgacgacaaatga

SEQ ID NO: 40 - protein, 525 aa, Artificial Sequence
  fusion protein derived from E6 and E7 proteins of human papilloma virus
MHQKRTAMFQDPQERPRKLPQLCTELQTTIHDIILECVYCKQQLLRREVYDFAFRDLCIVYRDGNPYAVDKLKFYSKISEYRHYCYSVYGTTLEQQYNKPLCDLLIRINQKPLCPEEKQRHLDKKQRFHNIRGRWTGRCMSCCRSSRTRRETQLGGGGGAAYMARFEDPTRRPYKLPDLCTELNTSLQDIEITCVYCKTVLELTEVFEFAFKDLFWYRDSIPHAAHKIDFYSRIRELRHYSDSVYGDTLEKLTNTGLYNLLIRLRQKPLNPAEKLRHLNEKRRFHNIAGHYRGQCHSCCNRARQERLQRRRETQVGGGGGAAYMHGDTPTLHEYMLDLQPETTDLYQLNDSSEEEDEIDGPAGQAEPDRAHYNIVTFCCKCDSTLRLCVQSTHVDIRTLEDLLMGTLGIVPICSQKPGGGGGAAYMHGPKATLQDIVLHLEPQNEIPVDLLQLSDSEEENDEIDGVNHQHLPARRAEPQRHTMLCMCCKCEARIKLWESSADDLRAFQQLFLNTLSFVPWCASQQ

SEQ ID NO: 41 - DNA, 1584 bp, Artificial Sequence
  nucleotide sequence encoding fusion protein derived from E6 and E7 proteins of human papilloma virus
atgcatcagaagcgaactgctatgtttcaggaccctcaggagcggccacgcaaactgcctcagctgtgcaccgaactgcagacaactatccacgacatcattctggaatgcgtgtactgtaagcagcagctgctgaggagagaggtctatgacttcgcttttcgcgatctgtgcatcgtgtaccgagacggaaacccatatgcagtcgataagctgaagttctacagcaagatctccgaatacaggcattactgttacagcgtgtacgggaccacactggagcagcagtataacaagcccctgtgcgacctgctgatcagaattaatcagaagcccctgtgccctgaggaaaaacagaggcacctggataagaaacagagatttcataacatccgaggacgatggaccgggcggtgcatgtcctgctgtagaagctcccggactcgacgagagacccagctgggcggaggaggaggagcagcttacatggcacgattcgaggaccctacccgaaggccatataagctgcccgacctgtgcacagaactgaatacttctctgcaggacatcgagattacatgcgtgtactgtaaaaccgtcctggagctgacagaagtgttcgagtttgctttcaaggacctgtttgtggtctaccgggattcaatccctcacgcagcccataaaatcgacttctacagcaggatcagggaactgcgccactactccgacagcgtgtacggggatacactggagaagctgacaaacactggcctgtacaatctgctgatccgactgcgacagaagccactgaacccagccgaaaaactgagacacctgaacgagaagagacggtttcacaatattgcaggccattataggggacagtgccatagttgctgtaatcgagccaggcaggaaagactgcagcgccgaagggagactcaagtcggcggaggaggaggagctgcatacatgcacggcgacacccccacactgcatgaatatatgctggatctgcagcctgagactaccgacctgtaccagctgaacgattctagtgaggaagaggacgaaatcgacggaccagcaggacaggcagagcctgaccgggcccactataatattgtgacattctgctgtaagtgcgattctactctgcggctgtgcgtgcagagtactcatgtcgacatccgcaccctggaggatctgctgatggggactctgggcatcgtcccaatttgtagccagaaaccaggcggcggcggcggagcagcttacatgcacggacccaaggctaccctgcaggacatcgtgctgcatctggaacctcagaatgagattccagtcgacctgctgcagctgagtgattcagaagaggaaaacgacgagatcgacggcgtgaatcaccagcatctgcctgctagacgggcagagccacagcgacacacaatgctgtgcatgtgctgtaagtgtgaagccaggatcaagctggtggtcgagtcaagcgccgacgatctgcgcgccttccagcagctgttcctgaatactctgtcatttgtcccttggtgtgcctcccagcagtga

SEQ ID NO: 42 - protein, 340 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIWLIFKSILFLPCLRKKILKIRHGWEDVTKINKTEICSQLKL

SEQ ID NO: 43 - DNA, 1026 bp, Homo sapiens
atggaatcacggaaggacatcactaatcaggaggaactgtggaaaatgaagccaagaaggaatctggaagaggacgactatctgcacaaggacaccggcgaaacaagtatgctgaaacgaccagtgctgctgcacctgcatcagactgctcacgcagacgagtttgattgcccctctgaactgcagcacacccaggagctgttcccacagtggcatctgcccatcaagattgccgctatcattgcttcactgacatttctgtacactctgctgagagaagtgatccaccccctggccaccagccatcagcagtacttctataagatccctatcctggtcatcaacaaggtcctgccaatggtgagcatcacactgctggccctggtctacctgcctggagtgatcgcagccattgtccagctgcacaatgggacaaagtataagaaatttccacattggctggataagtggatgctgactaggaaacagttcggactgctgtccttctttttcgccgtgctgcacgctatctacagcctgtcctatcccatgaggaggagctaccggtataagctgctgaactgggcttaccagcaggtgcagcagaacaaggaggacgcatggattgaacatgacgtgtggcgcatggaaatctacgtgagcctgggcattgtcggactggccatcctggctctgctggcagtgaccagtatcccttctgtcagtgactcactgacatggagagagtttcactacattcagagcaagctggggatcgtgtccctgctgctgggcaccatccatgcactgatttttgcctggaacaagtggatcgatatcaagcagttcgtgtggtatactccccctacctttatgattgccgtcttcctgcccatcgtggtcctgatcttcaagtccatcctgttcctgccttgtctgcggaagaaaatcctgaaaattcggcacggatgggaggatgtcaccaaaatcaataagactgaaatctgtagccagctgaagctttaa

SEQ ID NO: 44 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 45 - DNA, 543 bp, Homo sapiens
atgcaggccgagggcagaggcacaggcggatctacaggcgacgccgatggccctggcggccctggaattcctgacggacctggcggcaatgccggcggacccggagaagctggcgccacaggcggaagaggacctagaggcgctggcgccgctagagcttctggaccaggcggaggcgcccctagaggacctcatggcggagccgcctccggcctgaacggctgttgcagatgtggagccagaggccccgagagccggctgctggaattctacctggccatgcccttcgccacccccatggaagccgagctggccagacggtccctggcccaggatgctcctcctctgcctgtgcccggcgtgctgctgaaagaattcaccgtgtccggcaacatcctgaccatccggctgactgccgccgaccacagacagctccagctgtctatcagctcctgcctgcagcagctgagcctgctgatgtggatcacccagtgctttctgcccgtgttcctggctcagccccccagcggccagagaagatga